(19) United States
(12) Patent Application Publication
(10) Pub. No.: US 2005/0177237 A1
(43) Pub. Date: Aug. 11, 2005

(54) Spinal cage insert, filler piece and method of manufacturing

(76) Inventors: Ben Shappley, Germantown, TN (US); Timothy Pryor, Yardley, PA (US); John Blum, Somerset, NJ (US); Thomas J. Bradbury, Yardley, PA (US); Peter A. Materna, Metuchen, NJ (US); Jonathan McGlohorn, Pomaria, SC (US); Thomas George West, Lawrenceville, NJ (US); Sunil Saini, Plainsboro, NJ (US); Thomas James Hatcher, Burlington Township, NJ (US); Ali Cem Recber, Piscataway, NJ (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 10/966,109

(22) Filed: Oct. 18, 2004

Related U.S. Application Data
(63) Continuation-in-part of application No. 10/122,129, filed on Apr. 12, 2002.
(63) Continuation-in-part of application No. 10/837,541, filed on Apr. 30, 2004.
(60) Provisional application No. 60/512,514, filed on Oct. 17, 2003.
(60) Provisional application No. 60/512,417, filed on Oct. 17, 2003.
(60) Provisional application No. 60/569,921, filed on May 10, 2004.
(60) Provisional application No. 60/583,670, filed on Jun. 28, 2004.
(60) Provisional application No. 60/283,564, filed on Apr. 12, 2001.
(60) Provisional application No. 60/466,884, filed on Apr. 30, 2003.
(60) Provisional application No. 60/512,373, filed on Oct. 17, 2003.

(51) Int. Cl. A61F 2/44; A61F 2/46; B29C 67/04; A61F 2/28

(52) U.S. Cl. 623/17.11; 264/44; 623/23.63

(57) ABSTRACT

A spinal cage insert for a spinal cage is provided. The spinal cage insert has a shape suitable to be inserted into and fit closely in an interior of the spinal cage. The insert may comprise a member of the calcium phosphate family. The spinal cage insert may be made to a desired shape of porous ceramic, and it may include channels and/or surface features. Various shapes of filler pieces are also provided, wherein the filler pieces may be suitable to augment external regions of vertebrae which have been fused to each other so as to promote build-up of bone. The spinal cage insert and/or the filler pieces may be osteoconductive and may also contain osteoinductive substances or material. The articles may also contain cavities suitable for containing particles of demineralized bone matrix (DBM). Methods of use and methods of manufacturing the spinal cage insert and filler pieces are also provided.

### CROSS-REFERENCE TO RELATED APPLICATIONS

This application claims the benefit of U.S. Provisional Application No. 60/512,514, filed Oct. 17, 2003, U.S. Provisional Application No. 60/512,417, filed Oct. 17, 2003, U.S. Provisional Application No. 60/569,921,filed May 10, 2004, and U.S. Provisional Application No. 60/583,670, filed Jun. 28, 2004, the disclosures of each of which are herein incorporated by reference in their entireties.

### FIELD OF THE INVENTION

The invention pertains to devices that can be coupled to bones as part of a surgical repair of bone disorders such as spinal disorders.

### BACKGROUND OF THE INVENTION

Treatment of spinal disorders, such as for remediation of herniated or degenerated discs or other spinal problems, sometimes involves fusion of adjacent vertebrae to each other (arthrodesis). Spinal fusion involves inserting between vertebrae, in place of the normal intervertebral disc, material which may be a combination of structural material and material that promotes bone growth. A spinal cage is a rigid, frequently metal, article which is capable of mechanically connecting adjacent vertebrae. It typically has some internal void space through which bone may grow. It also typically has some perforations or openings in its walls for the same purpose. Existing suppliers include Medtronic Sofamor Danek and DePuy Acromed.

The hollow interior of the spinal cage is sometimes occupied by a filler suitable to encourage or assist the growth of natural bone into the interior. This may be termed a “spinal cage insert.” INFUSE® from Medtronic is a collagen sponge containing recombinant human bone morphogenetic protein.

Among the materials which have been packed into spinal cages as loose, amorphous or deformable filler have been collagen; allograft; bone chips obtained from the patient during surgery, either from the primary surgical site or harvested from another site; demineralized bone matrix (DBM); and ceramic in the form of granules. Beta-TCP has been used as an extender, used together with chips of the patient's own bone obtained in the course of the surgery.

Calcium phosphate materials are of interest at least because of their ready availability and the avoidance of possible disease transmission to the patient, and because of their chemical similarity to the inorganic component of natural bone, and in the case of tricalcium phosphate, because of its resorbability. To date, the use of ceramic as spinal cage filler material has been limited to loose materials such as granular materials. As such, the ceramic filler material has not had any designed-in macroscopic geometric features which might help encourage the ingrowth of natural bone. Ceramic material has not been formed into shaped rigid spinal cage inserts whose dimensions are closely matched to those of the spinal cage.

In spinal fusion procedures, sometimes it is not possible to place enough of either structural or bone-growth-promoting material immediately between vertebrae in the space formerly occupied by the disc. Accordingly, sometimes it is also desirable to build up bony mass on the external region of the vertebrae such as in the vicinity of the spinous process or transverse process. U.S. Pat. No. 6,719,795 discloses a polymeric graft of a generally cylindrical shape which may be placed near the transverse processes of vertebrae being fused. However, the article described in that patent is only a polymeric resorbable membrane rolled into a cylindrical shape, and so the body of the described device does not contain calcium phosphate which is known to be useful for facilitating bone ingrowth. The article described in that patent also has only limited control of its structure at various dimensional scales. Other surgical procedures which have reinforced the external posterior of vertebrae have generally done so with metal rods or instrumentation, which have of course been nonresorbable and incapable of hosting any bone ingrowth.

In general, with a rigid filler piece, it is useful for encouraging bone ingrowth if the filler piece has a patterning on those surfaces which touch native bone, and also it is useful for the filler piece to have channels through it. Porosity is also useful. However, except as described in commonly assigned patent application “Methods and apparatus for engineered regenerative biostructures such as hydroxyapatite substrates for bone healing applications,” U.S. application Ser. No. 10/122,129, docket number 900122.432, rigid filler material has typically been featureless block having isotropic structure and geometry, and the material has not offered the bone ingrowth advantages which are possible with surface features such as recesses and/or channels. Simple shapes have been made of porous TCP under the name Cerasorb® by Curasan AG (Kleinostheim, Germany), sometimes having holes drilled through them.

When manufacturing of porous synthetic materials such as beta tricalcium phosphate has included a sintering step, the sintering step has been found to be dependent on a number of processing variables. A frequent concern has been distortion of dimensions and shape during sintering, especially for large articles, because sintering of an article made initially from powder can involve shrinkage of overall dimensions by as much as 20%. Bone augmentation parts which are intended to be placed parallel to the spine can involve implantable parts whose dimensions may be as much as approximately 4 inches (100 mm). Such practical difficulties with sintering have made it difficult to sinter articles having relatively large overall dimensions.

Articles made of ceramic such as members of the calcium phosphate family are osteoconductive but are generally considered not to be osteoinductive. Osteoinductivity was demonstrated in demineralized bone matrix in 1965 by Urist, who showed that demineralized bone matrix has properties of stimulating the differentiation of bone progenitor cells into actual bone cells. Demineralized bone matrix (DBM) basically is a soft or spongy material, especially when it is wet. Accordingly, DBM has been made into a major component of putty, sheet, and other forms which have been flexible. A limited number of solid implant articles have been made by molding DBM with a binder. However, in general, adding DBM to an article could not be done simply by soaking an already-manufactured article in a liquid, because DBM is generally used in the form of particles greater than a certain minimum size, typically 100 micrometers. There are also some osteoinductive substances which can be added in the form of a liquid, such as bone morphogenetic protein, transforming growth factor beta, etc.

A combination of osteoinductivity and osteoconductivity is disclosed in US Pat. No. 6,695,882, which pertains to spinal fusion surgery. In that patent, it is described that a chamber in a dowel derived from natural bone allograft may be packed with an osteogenic material composition which is described as “including autograft, allograft, xenograft, demineralized bone, synthetic and natural bone graft substitutes, such as bioceramics and polymers, and osteoinductive factors.” However, the fact that this material is described as being packed into a chamber indicates that the material does not have definite form.

Elsewhere, the combination of osteoinductivity and osteoconductivity in structures has been accomplished in the sense of soaking a porous osteoconductive structure with an osteoinductive liquid, which occupies pores in the structure. The liquid has contained osteoinductive substances such as bone morphogenetic proteins. However, this approach has only been applicable to osteoinductive substances which are liquids. There has been no way to introduce DBM particles into porous structures because, as mentioned, there is a size which DBM particles have to have to be effective and even if such pores existed in structures those pores were inaccessible for introducing particles of DBM into them.

Until now, the need for osteoinductive additives to be strictly liquid has restricted the osteoinductive additives which could be used. Thus, basically there has been a limitation against the use of solid particulate osteoinductive materials as an additive to implantable structures. More specifically there has been no way to use DBM, which is an excellent osteoinductive material, in rigid osteoconductive structures.

Three dimensional printing may be used in the fabrication of materials. Three dimensional printing enables the control of the three dimensional shape of a fabricated material. Three dimensional printing is described in U.S. Pat. Nos. 5,204,055, and 6,139,574 and related patents, the subject matter of which are related to these patents.

Accordingly, in regard to spinal cage inserts, it would be desirable to provide spinal cage inserts having a defined shape which closely fits into and can be inserted into the interior of the spinal cage.

It would be desirable to provide spinal cage inserts having any of various geometric features useful for gripping the spinal cage insert, installing the spinal cage insert in the spinal cage, and retaining the spinal cage insert in the spinal cage.

In regard to augmentation on the exterior of vertebrae, it would be desirable to have a synthetic bone graft suitable for augmenting external regions of fused vertebrae in the region of the spinous process or the transverse process, as a way of providing increased mass of fused bone in the fused vertebrae.

For articles which involve substantial dimension in one direction, it would be desirable to make articles which in combination achieve a relatively large total length dimension while limiting the dimensions of any individual article so as to minimize problems during sintering.

In connection with either spinal cage inserts or filler pieces for use adjacent to vertebrae, the following would be desirable:

It would be desirable to provide articles which are at least partially resorbable so as to eventually be replaced by natural bone.

It would be desirable to provide articles which do not require harvesting of bone from the patient at a second site.

It would be desirable for the article to contain a substantial amount of calcium phosphates.

It would be desirable for the article to have a designed architecture at any of various dimensional scales.

It would be desirable for the article to include surface and/or internal geometric features suitable to promote the ingrowth of natural bone, such as channels or surface recesses.

It would be desirable to provide articles which are porous and which wick blood, platelet rich plasma, bone marrow or other bodily fluids, so as to promote ingrowth of natural bone.

It would be desirable to provide an article which is both osteoconductive and osteoinductive, by having a structure which is osteoconductive and which contains particles of DBM as the osteoinductive material.

It would be desirable for the structure to comprise members of the calcium phosphate family such as tricalcium phosphate.

It would be desirable for particles of DBM to be affixed in appropriate places (besides merely occupying such places) such that they do not readily move away.

It would be desirable to provide a place within the article for placement of substances such as DBM. It would be desirable in some cases to have closed ends so as to prevent such substances from leaving through the ends of the article.

It would be desirable for such an article to be able to be manufactured by three dimensional printing.

It would be desirable to provide a kit which includes a spinal cage insert and any of various other articles such as filler pieces for augmenting external vertebral surfaces; cutting tools and/or templates or other items needed to suitably modify bone; bone putty; and other surgical items.

### SUMMARY OF THE INVENTION

Accordingly, various embodiments of the invention are directed to a spinal cage insert for a spinal cage. The spinal cage insert has a shape suitable to be inserted into and fit closely in an interior of the spinal cage. The insert may comprise a member of the calcium phosphate family. The spinal cage insert may be made to a desired shape of porous ceramic, and it may include channels and/or surface features. Various shapes of filler pieces are also provided, wherein the filler pieces may be suitable to augment external regions of vertebrae which have been fused to each other so as to promote build-up of bone. The spinal cage insert and/or the filler pieces may be osteoconductive and may also contain osteoinductive substances or material. The articles may also contain cavities suitable for containing particles of demineralized bone matrix (DBM). Methods of use and methods of manufacturing the spinal cage insert and filler pieces are also provided.

### BRIEF DESCRIPTION OF THE FIGURES

FIG. 1 shows an axisymmetric bone void filler article comprising, or alternatively consisting of, a cylindrical bone void filler further comprising internal dead-end channels or surface indentations according to an embodiment of the invention.

FIG. 2 shows a spinal cage insert according to an embodiment of the invention.

FIG. 3 shows a filler article comprising a cylinder according to an embodiment of the invention.

FIG. 4 shows a cross-sectional view of the article of FIG. 3 according to an embodiment of the invention.

FIGS. 5-7 show a spinal cage insert having the shape of a container with a cap according to an embodiment of the invention.

FIG. 8 shows a cutaway of the article of the present invention containing particles of demineralized bone matrix in its interior.

FIG. 9 shows a strut according to an embodiment of the invention.

FIG. 10A shows an external view of a tubular filler piece having a stepped engagement feature according to an embodiment of the invention.

FIG. 10B shows a cross-section of the article of FIG. 10A.

FIG. 10C shows three of the articles of FIG. 10A engaged with each other according to an embodiment of the invention.

FIG. 11A shows an external view of a tubular filler piece having a sloped engagement feature according to an embodiment of the invention.

FIG. 11B shows a cross-section of the article of FIG. 11A.

FIG. 11C shows three of the articles of FIG. 11A engaged with each other according to an embodiment of the invention.

FIG. 12 shows a tubular filler piece having engagement features according to an embodiment of the invention.

FIG. 13 shows a cross-sectional view of a stack of tubular filler pieces having closed ends at the extremity of the stack according to an embodiment of the invention.

FIG. 14A shows a tubular filler piece having some holes in its side according to an embodiment of the invention.

FIG. 14B shows a cross-section of another tubular filler piece according to an embodiment of the invention.

FIG. 15 shows a spinous process.

FIG. 16 illustrates use of a rectangular prismatic filler piece which is installed directly in the region of the spinous process according to an embodiment of the invention.

FIG. 17A shows a normal vertebra viewed from above.

FIG. 17B shows a vertebra that has received a tubular filler piece on each side of the spinous process according to an embodiment of the invention.

FIG. 18 shows a general schematic of a 3DP manufacturing process according to an embodiment of the invention.

FIG. 19 shows an apparatus suitable for performing three dimensional printing.

### DETAILED DESCRIPTION OF PREFERRED EMBODIMENTS

Some embodiments of the invention may include bone void filler pieces of shapes suitable for implantation in a bone void that was surgically created or that otherwise resulted from other conditions such as disease or traumatic injury.

Some embodiments of the invention provide articles which promote bone growth. These articles may be placed in the interior of a spinal cage (spinal cage inserts).

Some embodiments of the invention provide bone-growth-promoting articles (e.g., filler pieces) which may be placed at exterior surfaces of vertebrae. These various articles can be specified and manufactured in terms of their material composition and also in terms of their geometry. Some embodiments are directed to methods of manufacturing such articles.

According to some embodiments, the spinal cage insert and/or filler piece (either of which may be referred to as an article) may be made of particles of a matrix material which are partially joined directly to each other. The article may be porous, having a porosity and a pore size distribution. One possible set of porosity and pore size distribution is described in “Bone void filler and method of manufacture,” U.S. application Ser. No. 10/837,541, docket number 900122.468, as having a peak in the pore size distribution at approximately 60 micrometers. Another possible porosity and pore size distribution is described in U.S. application Ser. No. 10/122,129, as having a peak in the pore size distribution at approximately 8 to 20 micrometers. The porosities and pore size distributions (and other related specifications) described in those applications are incorporated herein by reference in their entirety. Typical porosities in either of these cases may be in the range from approximately 40% to approximately 70%. These are not exact requirements, however.

The article may be made of a material and a geometry which are suitable to promote wicking into the filler piece of bodily fluids such as blood, platelet rich plasma, bone marrow or other fluids. Wicking of bodily fluids may be advantageous in promoting ingrowth of natural bone. For example, the porosity and pore size described herein are suitable to promote wicking of bodily fluids, which in some cases are not extremely different from water in their physical properties.

The article may also be of a hardness such that it can easily be carved, abraded, cut (e.g., with a knife), or otherwise have material removed from it during surgery. For example, the article may have cuttability and abradability properties which approximately resemble the properties of chalk (such as that used on blackboards, or the mineral chalk). Any surface (or multiple surfaces) of the article might be subject to shaping during surgery.

The article may be made of synthetic material such as ceramic, or any member of the calcium phosphate family. Specifically, the article may be made of or may comprise tricalcium phosphate, which is often biodegradable. In some embodiments, the tricalcium phosphate may be of a crystal structure which is either alpha tricalcium phosphate or beta tricalcium phosphate or both, in any proportion. For example, the tricalcium phosphate may comprise at least approximately 80% beta tricalcium phosphate and at most approximately 20% alpha tricalcium phosphate. Beta tricalcium phosphate is believed to have desirable resorption characteristics. Hydroxyapatite is another suitable member of the calcium phosphate family, which is nonresorbable.

The article may further comprise any of various bioactive materials, such as those described in U.S. application Ser. No. 10/122,129.

The article may further comprise a radioopaque marker, which may be resorbable.

The article may be sterile and may be packaged so as to maintain sterility.

These articles may further be described and specified by their geometry. For instance, an article may have any geometric shape, and an article may be manufactured according to any geometric specification, e.g., by use of three dimensional printing.

The invention includes a spinal cage insert which may have a geometry which is suitable to be inserted into and closely fit in the interior of a spinal cage.

The geometric relation between the spinal cage insert and the spinal cage may be such that the spinal cage insert may slide, by a translational motion (or other motion), into its designed position within the spinal cage. The translational motion may be along an axis or length of the insert or along another direction. Such a relation is possible, for example, when the interior of the spinal cage is of a generally cylindrical geometry or when the interior is of a generally tapered shape such as conical, or in general for any spinal cage interior geometry which does not trap the spinal cage insert inside the spinal cage (e.g., hold the insert so that the geometries of the cage and insert are such that the insert substantially cannot escape the cage). Within the category of insertion by translational motion, it is possible for the spinal cage interior and the spinal cage insert to be rotationally asymmetric, in which case only a limited number of positions of the spinal cage insert inside the spinal cage would be possible. Alternatively, it is possible for the spinal cage interior and the spinal cage insert to be rotationally symmetric, in which case the spinal cage insert could occupy any of many rotational angles (e.g., wherein at each rotational angle the insert has a substantially similar geometry). Yet another alternative is that the spinal cage insert and/or the spinal cage interior might have features of a helical nature. At least one helical feature may cooperate with another feature such that the spinal cage insert could be inserted into the spinal cage with a combination of rotational and translational motion (e.g., as in the motion of a screw). For instance, a helical feature may cooperate with another feature such that the insert may be threaded into place inside the spinal cage. If the spinal cage insert is designed for installation by a combination of translational and rotational motion, it may have a geometry substantially similar to a tapered screw or an untapered screw. It is also possible that the spinal cage insert could slide into place by a translational motion and then lock into place by a rotational motion, or vice versa.

The spinal cage insert may have any of a variety of features which can act to retain the spinal cage insert inside the spinal cage and possibly even create force between the spinal cage insert and the spinal cage. The existence of force by which the spinal cage insert bears against the interior of the spinal cage can be useful to allow easier installation of the spinal cage insert into the spinal cage and to prevent post-operative migration of the insert from the cage, and possibly even to improve the bone growth process. There are a variety of features and techniques that could be used to retain a spinal cage insert in a spinal cage. These features and techniques could be used singly or in combination, depending on the design of a particular spinal cage and/or the surgical procedure used to install the spinal cage.

One possible way of retaining a spinal cage insert in a spinal cage is by friction. The exterior of the spinal cage insert may have a close fit or a dimensional interference with a corresponding portion of the interior of the spinal cage. The close fit or dimensional interference may occur either with untapered shapes or with tapered shapes. The spinal cage insert may be manufactured with a taper, which may or may not correspond to a taper in the spinal cage interior. Either substantially all of the exterior surface of the spinal cage insert may be involved in fit/interference, or else only some features on the exterior surface may be so involved. For example, features such as knurling, ribs or protrusions may be incorporated into the design of the insert, such that only those features have a close fit or interference. It is further possible that the spinal cage insert may be designed so that it, or appropriate features of it, can deform, crush, or be sheared off upon installation. This could accommodate a wider range of tolerances for the manufacture of the insert than would be possible without planned crushing. This could be useful if, e.g., only isolated features were required to crush, rather than the entire surface of the spinal cage insert. As the spinal cage insert is pressed into the spinal cage, a variety of methods may be used to ensure that sufficient friction forces may be developed to keep the spinal cage insert in place within the spinal cage. Many of these methods are known to those skilled in the art.

Another possibility is that the spinal cage insert may be made in at least two parts, or be capable of separating into at least two parts. At an appropriate time after installation of the spinal cage insert into the spinal cage, the parts may be separated from each other by a separator. The shape of the separator may be substantially planar, e.g., as in the case of a wedge having two nearly opposing surfaces. Other examples include a screw and other shapes having some rotational symmetry. In any event, the spinal cage insert may then expand within the spinal cage upon insertion of the separator. The choice of shape would depend in part on the geometry of the spinal cage. Any expandable spinal cage insert may have protrusions on the exterior suitable to engage corresponding features on the interior of the spinal cage, so that the spinal cage insert may be captured within the spinal cage. The spinal cage insert may be fabricated as multiple subcomponents, or may be fabricated as a single article which is capable of fracturing into multiple subcomponents as the separator is inserted. In the latter case, creating cracks or thin sections within the spinal cage insert to act as stress-raisers, is one possible way to determine the location of a fracture in the spinal cage insert.

Yet another possible way of retaining a spinal cage insert in a spinal cage is by using a geometry involving one or more locking components, whereby the spinal cage insert is trapped by the placement of the locking component(s). An example is the insertion of a pin through a hole through the wall of the spinal cage and a corresponding hole in the spinal cage insert. The axis of insertion of the locking component may be substantially perpendicular to that of the spinal cage insert, although it does not have to be. The locking component(s) may be placed after the spinal cage insert is within the spinal cage. The assembly of the spinal cage insert and the spinal cage may then be placed in the spine, such that the pin is in contact with the vertebral bodies. The vertebrae may trap the pin in the spinal cage, and the pin would in turn hold the spinal cage insert in place in the spinal cage. The pin may be made of a material similar to the spinal cage insert and may contain any or all of the features of the spinal cage insert itself.

There are still other geometric features which the spinal cage insert may have. It is possible that a surgical procedure for installing the spinal cage insert may involve installing the empty spinal cage in the spine and then placing the spinal cage insert within the spinal cage. At the time the spinal cage insert is inserted into the spinal cage, there may be problems of limited access and/or poor visibility at the surgical site. It may therefore be desirable to include in the design of the spinal cage insert one or more guiding features suitable to assist in the installation of the spinal cage insert within the spinal cage. Such a guiding feature may be useful even if the spinal cage insert is installed in the spinal cage at an earlier time. One such guiding feature which the spinal cage insert may have is a chamfer or taper, whereby the leading edge of the spinal cage insert has a loose fit within the spinal cage suitable to guide the trailing, closer-fitting portions of the spinal cage insert into the spinal cage. Insertion of the spinal cage insert into the spinal cage would have been more difficult to achieve without the taper or guiding feature. One specific example of a guiding feature is the fit of a conical portion of a spinal cage insert into a spinal cage which contains a cylindrical or conical interior surface.

Another possible geometric feature of the spinal cage insert is a carrying feature suitable to allow the spinal cage insert to be gripped or carried at the time the spinal cage insert is inserted into the spinal cage. Such a feature may cooperate with an appropriate tool. Such a feature may, for example, include recessed or flat surfaces which may cooperate with a tool such as tweezers. Such a feature may, for example, be a hole which extends some distance into the spinal cage insert. The hole may have a non-round cross-section and may cooperate with a similarly-shaped tool. Such a shape and corresponding tool would provide control over the angular orientation of the spinal cage insert during insertion, and could be used to rotate the spinal cage insert if the insertion required any rotational motion.

The spinal cage insert may have a plurality of negative geometric features (i.e., recessed or internal features which suggest that material is in some sense “missing” from an overall shape, such as a hole) at any one or more of its surfaces. Negative features may be considered to be any form of missing material feature which occupies a minority of the surface area. (Features which would be considered positive surface features are also possible and are discussed elsewhere herein, e.g., crushable ribs.) Negative features can be dead-ended recesses or can be channels which go through the spinal cage insert to exit at a surface of the spinal cage insert. Negative features may also comprise hollow portions within the general shape of the insert. In general, any surface or combination of surfaces may be provided with such negative features.

The distribution of such features can in general be of any pattern on any surface or combination of surfaces of the spinal cage insert. Such surface recesses or channels may have a smallest dimension, e.g., along a surface of the spinal cage insert, which is in the range from approximately 50 micrometers to approximately 3000 micrometers. Other lengths and ranges may be considered.

It is further possible, assuming that the spinal cage has perforations or openings, that the locations of the surface recesses or channels in the spinal cage insert could be coordinated with the location of the perforations or openings through the spinal cage. For example, it may be desirable that surface recesses or channels be located so as to substantially coincide with the perforations or openings through the spinal cage insert. In this way, tissue could grow through the perforations or openings in the spinal cage and then further grow into the surface recesses or channels in the spinal cage insert. Such a system of passageways (perforations/openings plus recesses/channels aligned with each other) may provide one or more pathways by which new tissue may enter the spinal cage insert before the new tissue growth branches out to enter individual pores of the spinal cage insert. The surface recesses or channels may, for example, be helpful for establishing vascularity to support new tissue growth. Ensuring that the position of surface recesses or channels is aligned with the position of perforations in the spinal cage is especially possible if the geometries of the spinal cage interior and of the spinal cage insert are not rotationally symmetric. This would limit the number of possible relative insertion positions to a small number. Ensuring such alignment is also possible in the case of rotational symmetry, if at least some non-symmetric feature(s) is provided in either the spinal cage or the spinal cage insert to define the relative angular orientation of the spinal cage and the spinal cage insert.

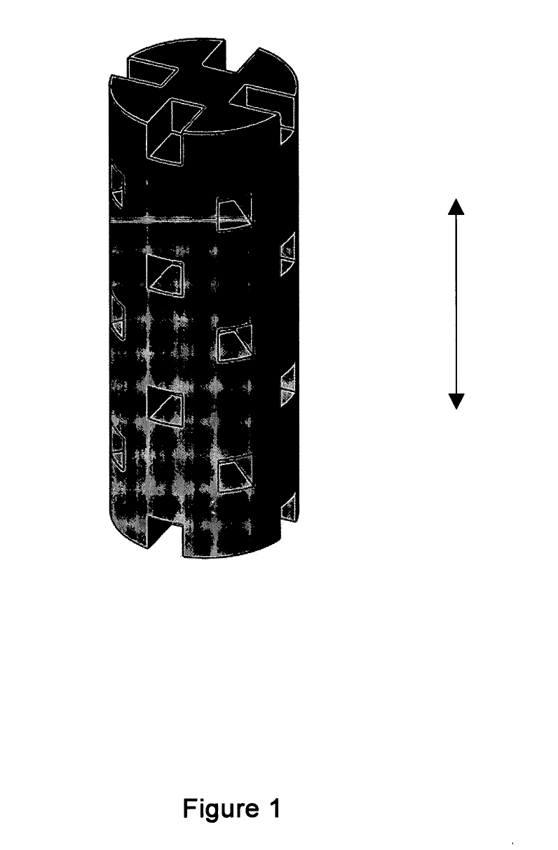

FIG. 1 shows a generally cylindrical spinal cage insert according to an embodiment of the invention. A cylindrical insert may be appropriate for a spinal cage whose interior is of a similar shape. The insert may comprise, or alternatively consist of, a cylindrical bone void filler further comprising internal dead-end channels or surface indentations. The features at the ends of this embodiment of bone void filler article may be suitable to engage tooling (such as tooling for handling the bone void filler article or for inserting the bone void filler into a surgical site).

The arrow shows the direction in which the insert can be inserted into the spinal cage. Here, the direction is a translational direction along the length direction (and axis direction) of the insert.

In this embodiment, the negative features are illustrated as surface recesses which are dead-ends and do not intersect other surface recesses. This may be accomplished in part by having recesses be located at places which are staggered along the axial direction of the generally cylindrical geometry. In this embodiment the surface recesses are distributed in a symmetric pattern, although this is not necessary. In other embodiments, it is possible that the spinal cage insert could have negative features such as channels that transit completely through the spinal cage insert or recesses that intersect with other recesses.

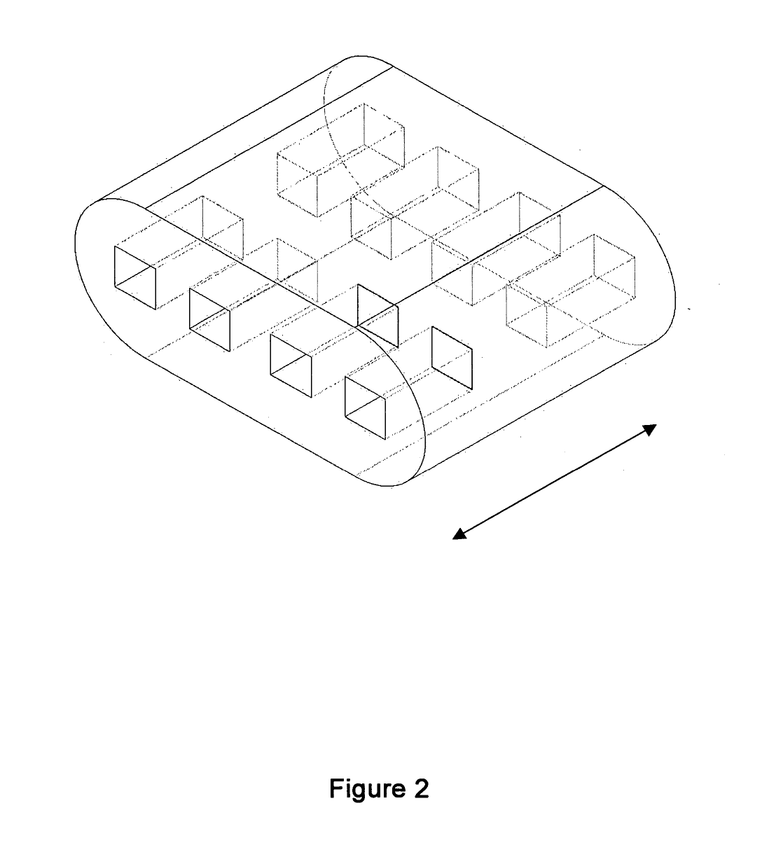

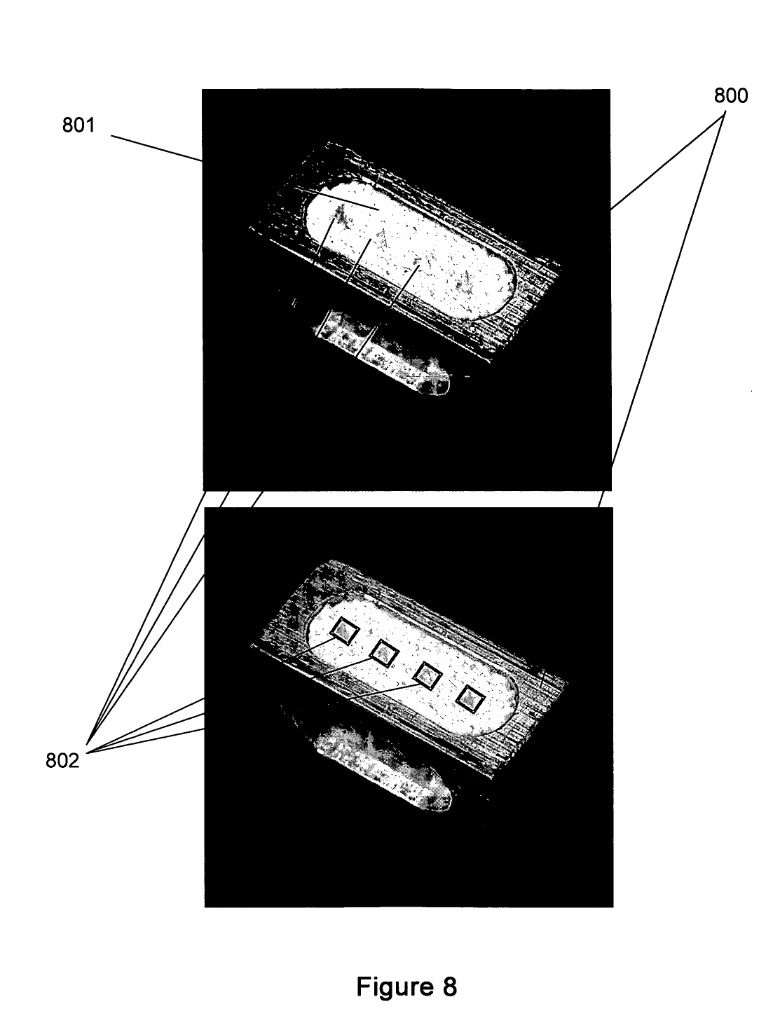

In some embodiments, the spinal cage insert may have a shape as shown in FIG. 2. This design illustrates a spinal cage insert suitable for use in a spinal cage which could be described as an open box (e.g., see FIG. 8), having an interior which is not rotationally symmetric. The direction of insertion of the spinal cage insert into the spinal cage may be as shown in the illustration. In this illustration, the recessed surface features are shown as being on surfaces which can be described as the front and back of the spinal cage insert (with respect to the motion for sliding the spinal cage insert into the spinal cage). In general, any surfaces or combination of surfaces could have recesses or other surface features.

The arrow shows the direction in which the insert can be inserted into the spinal cage. Here, the direction is a translational direction along the length direction (and axis direction) of the insert.

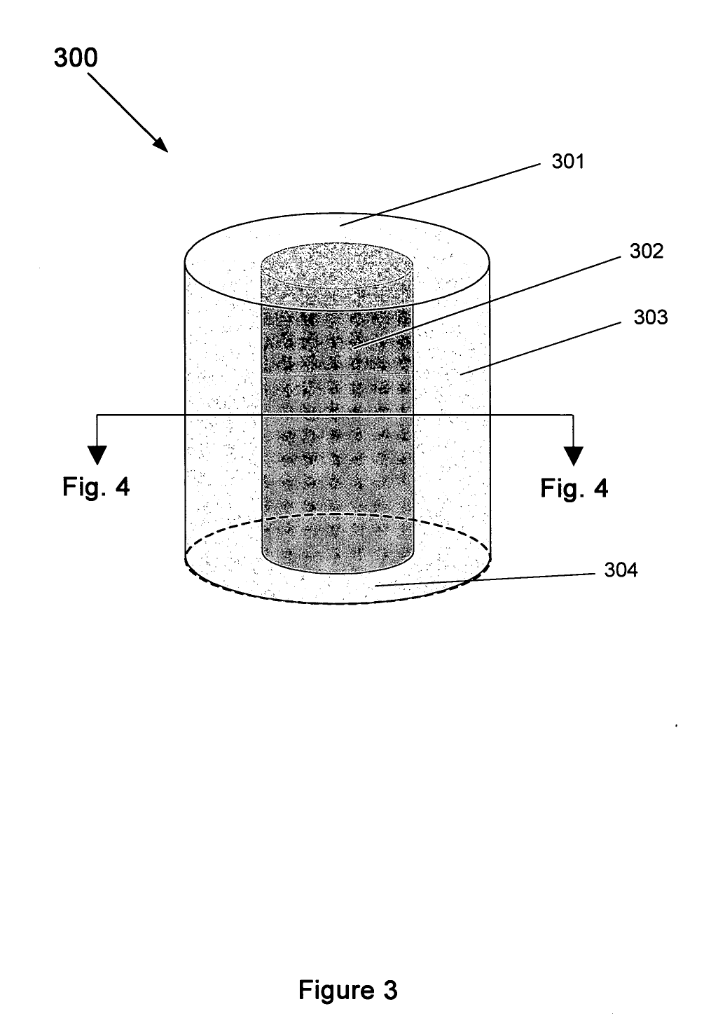

FIG. 3 shows a filler article 300 comprising a tubular cylinder 303 which may have one or more closed ends 301, 304 according to an embodiment of the invention. An inner portion 302 defined by the interior of the tubular portion 303 may contain DBM or other materials. The one or more ends 301, 304 may be closed (or open). If they are closed, they may enclose the DBM or other materials inside the tubular cylinder 303.

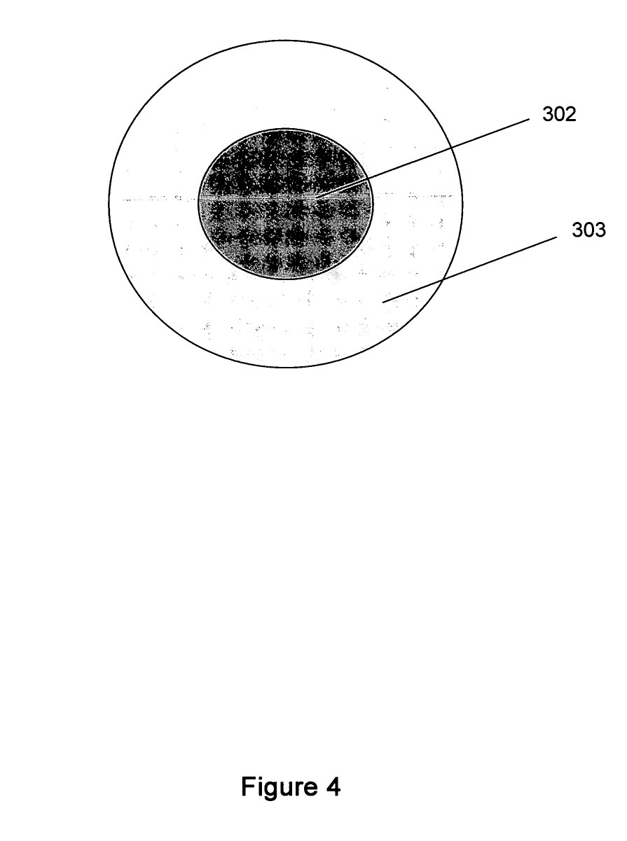

FIG. 4 shows a cross-sectional view of the article of FIG. 3 according to an embodiment of the invention. FIG. 4 shows the tubular cylinder 303 enclosing the inner portion 302.

Articles having cavities may contain particles of DBM. These articles may comprise caps which help to retain the particles of DBM inside the article. Such caps may comprise gelatin, for example, and a portion of the cap may interpenetrate with (or otherwise be coupled to) the article itself. Such a cap may be applied when it is in the form of a gel or thick fluid, which may harden. Thus, the cap may be made at the time of use, and it may have a geometry determined according to the needs of its use. For instance, it may be fitted to a container of a variety of sizes.

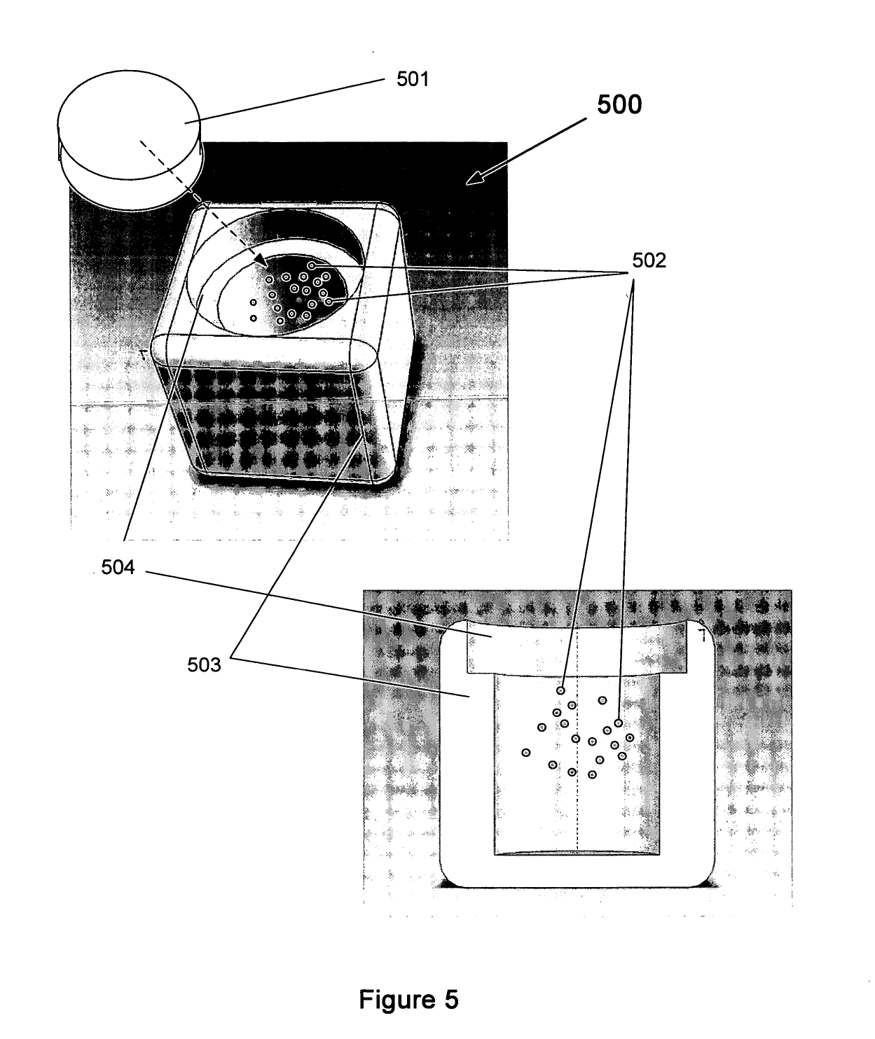

FIGS. 5-7 show a spinal cage insert 500 having the shape of a container 503 with a cap 501 according to an embodiment of the invention. DBM or other materials 502 may be placed inside the interior of the container 503. A cap 501 may be placed on the container 503, thereby enclosing the DBM and/or other materials 502. The cap 501 may be porous or solid, and it may comprise shapes and geometries as described herein. It may comprise a substantially similar material as that described for spinal inserts. It may also comprise a gel or other material.

One advantage of using a cap is that the container and cap may be manufactured separately, before any agents (e.g., DBM) enter the container. Many methods of manufacturing involve heating, which may have disadvantageous effects on such agents. Thus, the container and cap can be manufactured, agents can be inserted into the container, the cap can be coupled to the container to enclose the agents, and the composite article may be used for a medical procedure; e.g., the article may be coupled to a bone or inserted into a spinal cage, which may then be coupled to a bone (e.g., a spine).

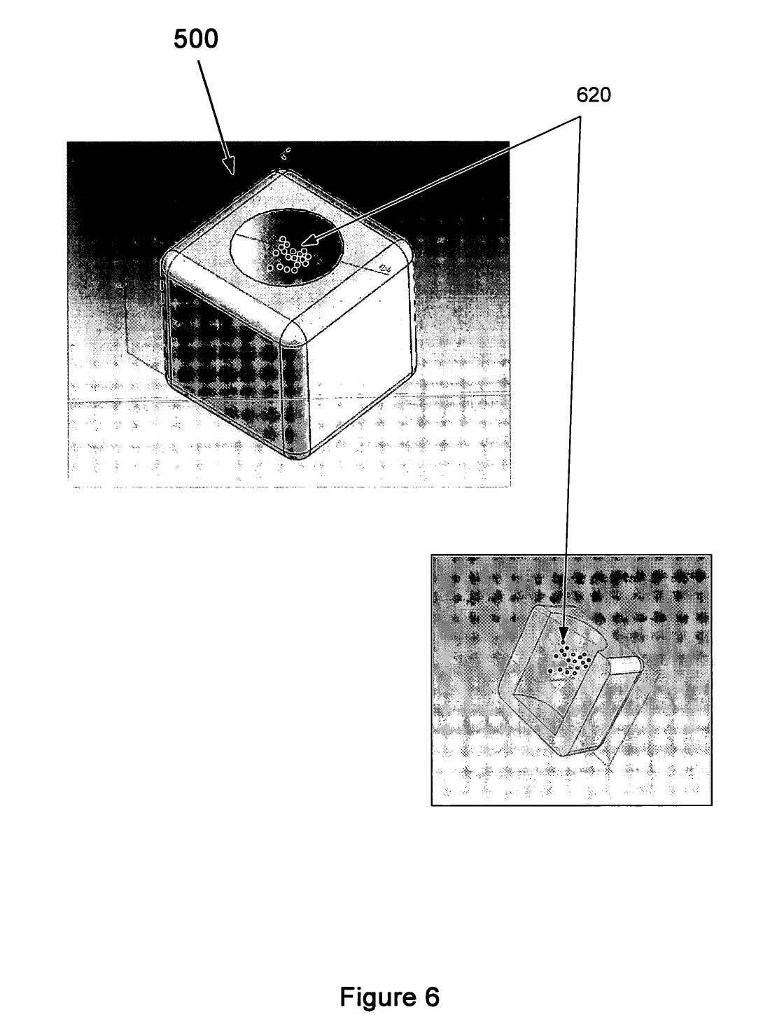

FIGS. 5 and 6 show DBM and/or other agents in the interior region of an insert. It should be noted that the DBM may be accompanied by other agents. The structure may have multiple channels, and some or all of the channels may be filled with DBM. It is believed in the art that in order for particles of DBM to exhibit osteoinductivity, those particles should be larger than approximately 100 micrometers. Channels filled with DBM may have dimensions of at least 0.5×0.5 mm, and that channels which are not intended to be filled with DBM may have dimensions smaller than the channels which are intended to contain DBM. For example, the non-DBM-filled channels may have cross-sectional dimensions as small as 0.1 mm×0.1 mm. The channels which are too small to hold particles of DBM may still be provided for the purpose of conducting the ingrowth of tissue or providing place for blood vessels to grow. Such features are believed to be helpful for promoting ingrowth and integration. Channels may or may not traverse completely through the structure, i.e., channels may be either open-ended or closed-ended (dead-ended). Also the channels may lie along various different planes or have different directions, in any relative combination and orientation. The cross-sectional shape of the channels may be cylindrical, rectangular, or another shape.

The container 503 may comprise a cap insertion portion 504 which can couple to the cap 501, e.g., by friction forces. For instance, the cap insertion portion 504 may have substantially similar dimensions as the cap 501, so that the cap 501 can be coupled to the cap insertion portion 504 by friction forces. Any coupling means or geometries may be used as described herein. The cap 501 and container 503 may comprise any container and cap as known in everyday household items, such as bottlecaps, screw-on lids. For instance, the cap 301 may comprise a tab as used in battery covers of remote control devices.

FIG. 6 shows an article which contains in its interior a plurality of particles 620 of a substance such as demineralized bone matrix in a tubular filler piece 500. The article may also comprise, in its interior, bone chips or any other suitable growth-promoting substance. FIG. 6 also shows a cross-sectional view of the filler piece 500.

Within the pores defined by the porous osteoconductive material, there may be other substances such as bioactive substances. Bioactive substances are discussed in commonly assigned co-pending U.S. application Ser. No. 10/122,129, filed Apr. 12, 2002.

The article may be sterile and may be packaged appropriately to maintain sterility until the time of use.

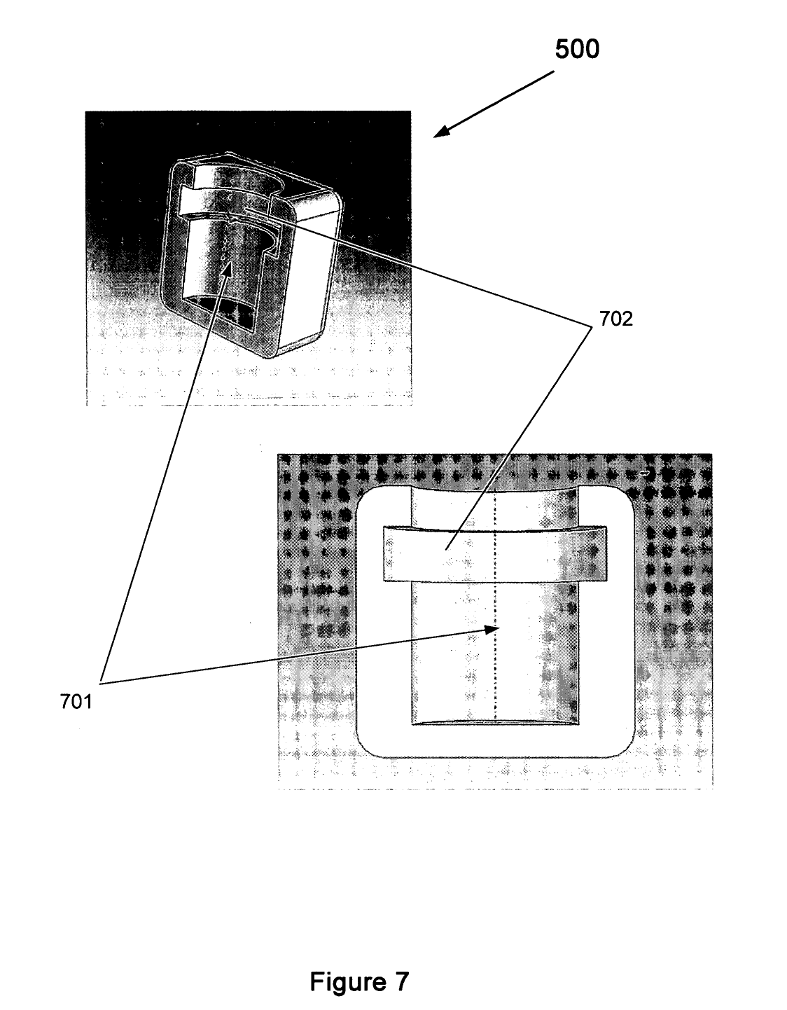

FIG. 7 shows a filler piece according to an embodiment of the invention. The filler piece may comprise an interior region 701 suitable for containing materials such as DBM. The filler piece of FIG. 7 is open at the top and closed at the bottom. The filler piece may also comprise a cap insertion portion suitable for engaging with a cap. The cap may substantially enclose certain materials inside the filler piece, such as DBM particles.

FIG. 8 shows two photographs of a spinal cage coupled to a spinal cage insert according to an embodiment of the invention. The insert comprises recesses 802. The light-colored inner material is the insert 801. The darker-colored outer portion is the spinal cage 800, which may comprise metal. The top photograph of FIG. 8 has not been modified. The bottom photograph has been modified to highlight the location of four recesses 802 to make them easier to see.

In some embodiments, the insert should be dimensionally matched to the spinal cage. For instance, the one or more inserts should be of an approximate size and shape of (e.g., slightly smaller than) the interior region(s) of the cage. An insert that is too small may slip out of a cage. An insert that is too large may be difficult (or impossible) to place into the cage (e.g., during surgery). In some embodiments, a relatively loose-fitting insert may be preferred, wherein the insert may be large enough to remain inside the cage (and not slip out), but small enough that fluids (e.g., bodily fluids) can pass easily between the insert (or portions thereof) and the cage (or portions thereof). FIG. 8 shows such dimensional matching of cage and insert.

In terms of more localized geometry within the article, the article may be porous, and may comprise particles which are partially joined to each other but still leave some space between themselves in the form of pores. The pores may be characterized by pore sizes which may be in the approximate range of 1 micrometer to 1000 micrometers. Other sizes may be considered. There may be an average pore size of approximately 60 micrometers. Average pore sizes of approximately 20, 40, 80, 100 micrometers, or another dimension may also be considered. The article may be osteoconductive and may be made of appropriate materials as described elsewhere herein.

The article may further comprise spaces in its interior such as macroscopic channels or macroscopic interior voids. The macroscopic channels may have cross-sectional dimensions, or the macroscopic interior voids may have dimensions which are greater than approximately three times (or two times or ten times) the average pore diameter, so that the feature is distinguishable as being larger than a pore. Other relative sizes may be considered. Further, the macroscopic channels and macroscopic interior voids may have dimensions which are greater than the dimensions of usefully sized particles of DBM, as described elsewhere herein. The channels may comprise channels open at both ends, blind channels, surface features resembling tire treads, straight channels, channels with curves or changes of direction, constant-cross-section channels, tapered channels, intersecting channels, macroscopic void spaces connected by at least one channel to the exterior, or other types of channels.

In some embodiments, the macroscopic channels or macroscopic interior voids may have access to (or other connection to) the exterior surface of the structure. As described elsewhere herein, this may be useful for placing the osteoinductive material such as DBM inside those channels or voids. FIGS. 2-7 show some simple shapes of inserts which include channels or recesses or internal space for containing DBM. Such an article may also contain a cap or caps to cover openings and thereby retain particles of DBM or any other substance placed in an interior region of the article.

FIG. 8 shows a spinal cage 800 and spinal cage insert 801 according to an embodiment of the invention. The insert 801 of FIG. 8 may comprise a geometry substantially similar to that shown in FIG. 2. As shown in FIG. 8, the insert 801 may fit inside the cage 800. Due to the closeness of the fit, friction forces of the insert 801 against the inner surface(s) of the cage 800 may prevent the insert 801 from leaving an inner portion of the cage 800.

The invention also includes varieties of filler pieces which may be suitable for use in augmenting external surfaces of vertebrae.

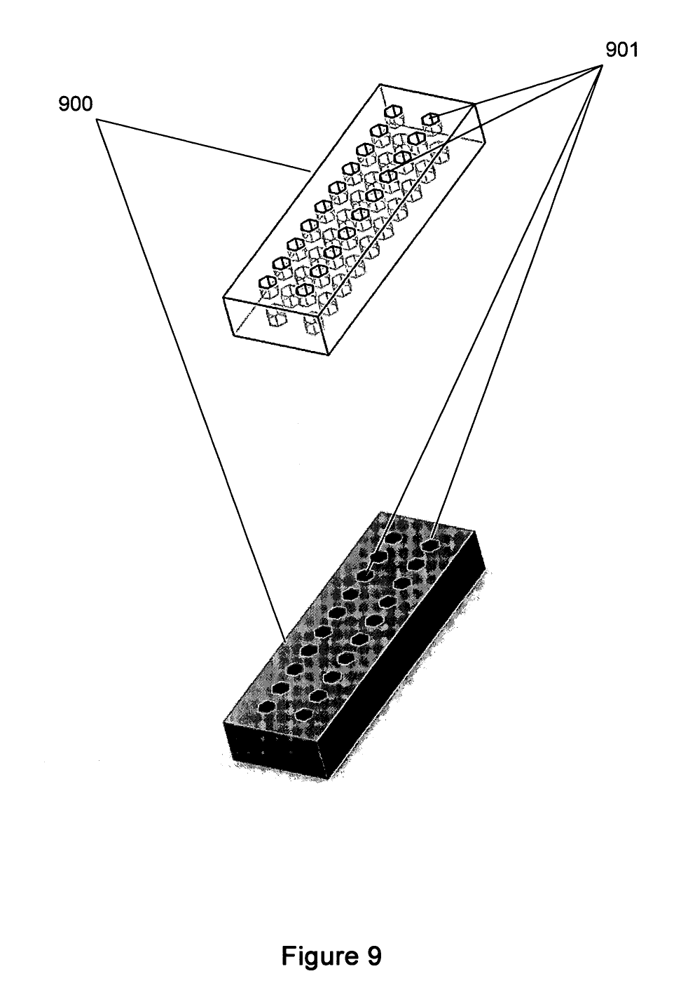

FIG. 9 shows a filler piece according to an embodiment of the invention. One such possible shape of a filler piece is substantially a rectangular prism, as illustrated in FIG. 9. While the insert may have a general shape (such as a rectangular prism), it would also be possible for one or more edges of the insert in FIG. 9 to be rounded or otherwise modified. Although the filler piece has been shown with surfaces which are flat planes, it is not necessary for the surfaces to be perfectly flat or for the filler piece to be a perfect rectangular prism.

Some embodiments provide for a cylindrical or tubular filler piece article that is shaped suitably to augment an external surface of vertebrae. The invention may similarly include more than one cylindrical or tubular article which are suitable to fit into each other end-to-end. A tubular article may be defined in part by an external surface and an internal surface. The external surface may be generally cylindrical or may be some other axisymmetric shape. The internal surface may be cylindrical or may be some other shape (e.g., an axisymmetric shape). If both the external surface and the internal surface are axisymmetric, the two surfaces may be substantially coaxial with each other (or not). If the geometry does not possess axisymmetry, the external surface and the internal surface still may define a region (e.g., a wall) between them which may have an approximately uniform thickness. In general, the external and internal surfaces could be of any cross-sectional shape. Possible axisymmetric surfaces include cylindrical surfaces and also portions of cones, paraboloids and similar surfaces of rotation.

The article may be dimensioned, such as in its outside diameter or external surface dimensions, so as to fit in a concave region on either side of the spinous process of vertebrae or between the spinous process and the transverse process. For example, the article may have an outside dimension of approximately 12.7 mm (0.5 inch). The article may also have an outside dimension of 5 mm, 20 mm, or another size.

The article may further be dimensioned, in its internal dimensions such as inside diameter, so as to allow placement of further growth-promoting material in the interior. Such material may include DBM, which may typically exist in the form of particles of a known size, such as approximately 100 to 800 micrometers. The internal dimensions of the article may be sufficient to accommodate a desired volume of such substance, and to allow such substance to be placed into the interior of the article. For example, the article may have an inside diameter of approximately 6.35 mm (0.25 inch). The internal dimensions may be chosen appropriately to provide a wall thickness of approximately 3.2 mm (0.125 inch) (or other thickness).

The article may have a length such that a desired length (such as the height of a desired number of vertebrae) may be made up by two of these articles laid end-to-end, or some other small integer number of these articles. A typical desired total length might be approximately 100 mm (4 inch). It is possible that a single article of that length might be made. Alternatively, a length of an individual article might be slightly over 50 mm (2 inches) so as to fill that length using two articles, while providing some overlap between adjacent articles. Alternatively, a length of an individual article might be slightly over 33 mm (1.3 inch) so as to fill that length using three articles, while providing some overlap between adjacent articles.

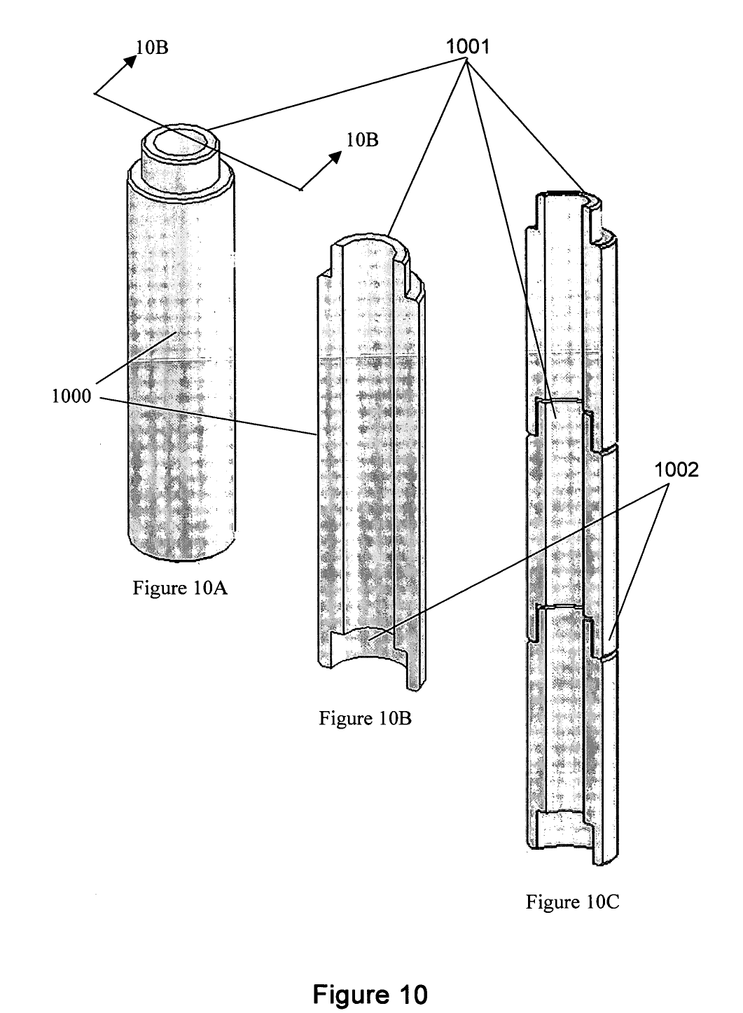

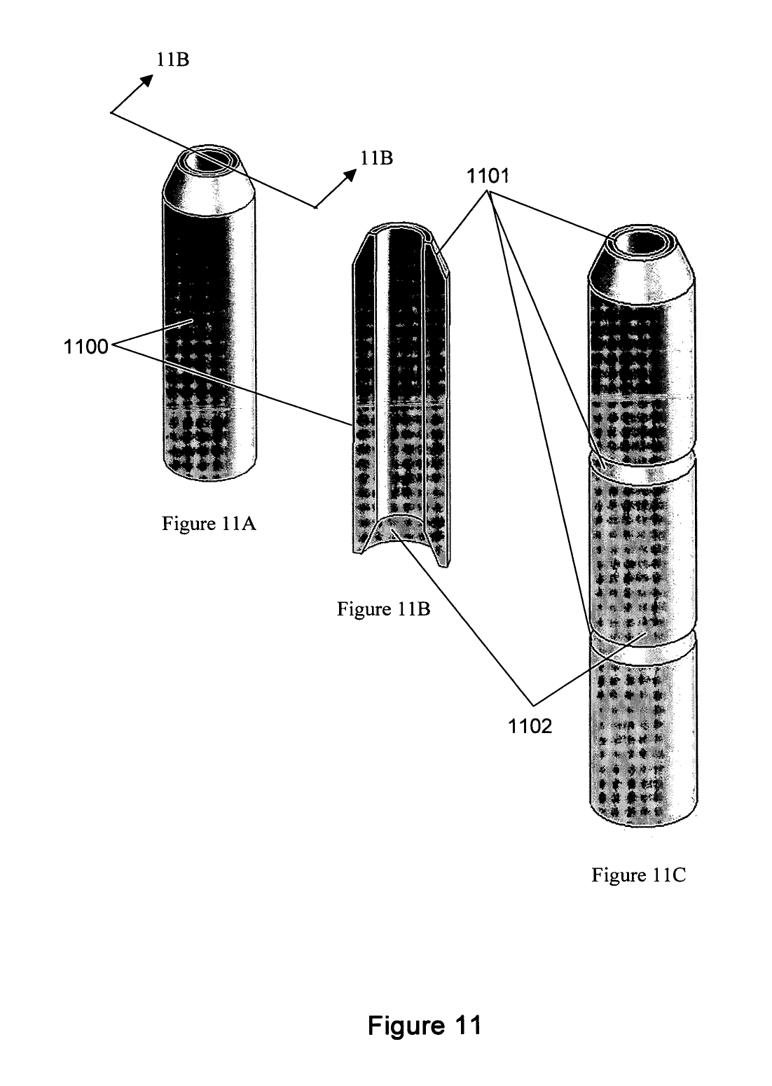

For the situation in which more than one of the articles are used to engage each other, the ends of the article which engages another may contain features suitable for engaging with corresponding features on the other article. These features may have any of a variety of designs. Some possible geometries involve axisymmetry of the engaging features. Examples are shown in FIGS. 10 and 11.

FIGS. 10 and 11 show tubular articles 1000, 1100 according to an embodiment of the invention. According to some embodiments of the invention, one or more articles (e.g., filler pieces or inserts) may be coupled to one another. The articles 1000, 1100 may fit on top of each other to make a longer composite coaxial tubular article, as shown in FIGS. 10C and 11C. The articles may be configured to engage in a manner similar to Lego™ pieces, Lincoln Logs™, Tupperware™ bowls and caps, stacks of cups, or other articles which can couple to one another. In some embodiments, it may be useful to have an coupling portion 1001, 1101 (e.g., a “male” portion) of one article engage a receiving portion 1002, 1102 (e.g., a “female” portion) of another article.

An external view of an article is shown in FIG. 11A. FIG. 11B shows a in cross-sectional view of the article of FIG. 11A. FIG. 11C shows multiple articles of FIGS. 11A and 11B in coupled form. Here, three tubular articles are coupled in coaxial form, wherein a narrow top portion of one article fits into a wider base portion of another article. The article accordingly has an engagement feature which is a stepped cylindrical geometry. The article has a main body portion which is generally cylindrical, having a main body outside diameter. Extending from one end of the main body is an engagement region having an engagement outside diameter which is less than the main body outside diameter. The engaging region may have an engaging region inside diameter which may be substantially equal to the main body inside diameter. The other end of article (or the end of the engaging article) may have an outside diameter substantially equal to the main body outside diameter and may have an engaging region inside diameter which is suitable to receive the engaging region outside diameter of the engaging end of the engaging article.

It is also possible that the engaging features can be such as to help to guide respective articles into the desired position relative to each other, even if the articles are not initially in such alignment. Such features may be helpful in view of limited working room or visibility at a surgical site. Such a design is shown in FIGS. 11A, 11B and 11C. An engaging pair of articles may comprise an inserting article and a receiving article. For example, one article (the inserting article) of such a pair of articles may have an end which is chamfered or tapered or frusto-conical as shown. The receiving article may contain an end which contains a concave feature. The concave feature on the receiving article may be an inverse of the chamfered or tapered or frusto-conical feature on the inserting article. However, it is also possible that the feature on the receiving article could be something other than an inverse of the feature on the inserting article and yet could still have usefulness in guiding the articles toward engagement with each other.

It is also possible that the engaging feature may be a cylindrical region of smaller diameter than the article, made of the same porous material as the rest of the article without a central hole through it. This engaging feature may have an outside diameter which is suited to fit into a corresponding inside diameter of another piece. This is shown in FIG. 11. Another possibility is that the engaging feature may be a coaxial post which does not have a macroscopic hole through it. Such a design also serves to close one end of the tubular filler piece helping to retain contents in the interior of the tubular filler piece. Such a post may be configured so as to fit inside a corresponding hole in another filler piece.

A still further possibility is that the engaging features may be nonaxisymmetric. For example, the interlocking features may be a pair of teeth or fins on each article.

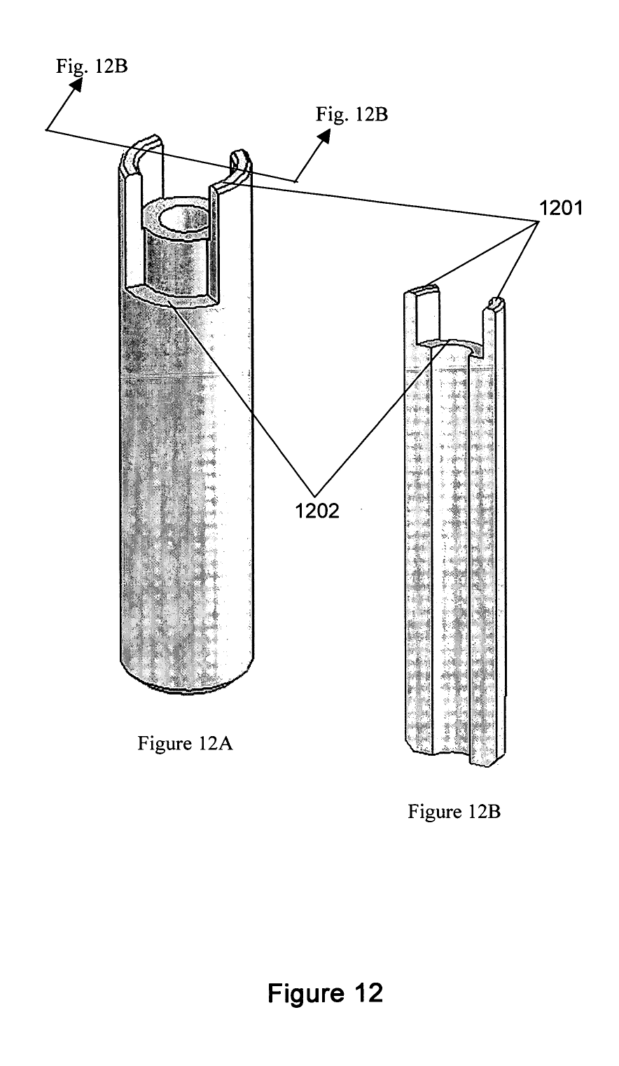

FIGS. 12A and 12B show an article according to an embodiment of the invention. The article comprises teeth 1201 and a receiving portion 1202 configured to receive (or otherwise couple with) the teeth of a similarly configured article. Each tooth may, for example, occupy no more than about 25% (or 10% or 50%) of the circumference of the article, so that the mating of adjacent articles is possible with teeth from one article interlocking with teeth from the other article. The two teeth on each article may be symmetrically opposed to each other.

The filler piece can have cross-sections other than circular, such as for example oval (e.g., hollow oval) or even rectangular (e.g., hollow rectangular), possibly with rounded corners. The filler piece could have at least one closed end, which may aid in retaining potentially loose or migratory material within its interior.

Still other designs of engaging features are also possible. Any set of shapes that can substantially couple to each other are considered herein, particularly shapes whose basic geometry immediately suggests the method and relative orientation of coupling.

When more than one tubular article is used, the individual articles can be identical to each other or can be different from each other. Articles which are at an end of the stack do not need to have engaging features at those ends which do not engage with other pieces, although they could have such features. It is possible that an end of an article which is at the end of a stack can be closed rather than open, to help contain a substance which may be placed inside the coupled (e.g., stacked) composite article. The last article in a series may comprise a closed end at one or both ends of a stack of articles or an individual article.

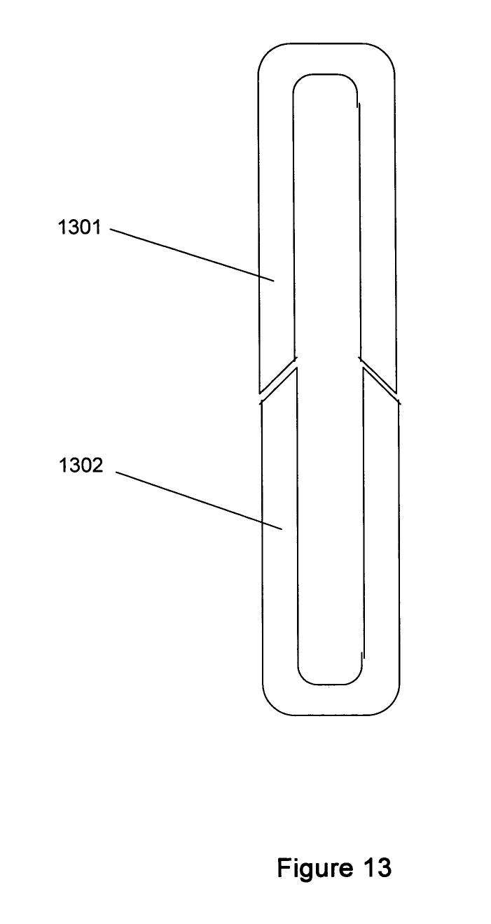

FIG. 13 shows a stack of two articles 1301, 1302 according to an embodiment of the invention. The articles have closed ends at the extremities of the stack. Such closed ends may help to reduce the migration of loose or migratory substances (e.g., DBM) which are place within the interior of the article.

Any segmented design of filler piece may be designed so that the individual filler pieces have a length which is more suitable for sintering than would be the overall combined length of a number of such articles. Such a length may be, for example, less than approximately 50 mm (2 inches) (or 25 mm, 100 mm, or another length).

The filler piece may have a defined local surface geometry in at least some surfaces. The defined surface geometry can exist on some surfaces and not on other surfaces. The defined surface geometry may comprise surface recesses such as dimples, depressions, grooves, etc., suitable to promote ingrowth of natural bone. The defined surface geometry may comprise channels, extending through the filler piece from one surface to another surface, or extending in any other geometry including dead-ended channels, suitable to promote ingrowth of natural bone. Channels may be of any cross-section including round, rectangular and other cross-sectional shapes. Such surface recesses or channels may have a smallest dimension, along a surface of the filler piece, which is in the range from approximately 50 micrometers to approximately 500 micrometers. The filler piece may have both channels and surface recesses, in any combination, on any surface. For example, it is known that for physiological reasons, there is a maximum distance, typically 2 mm, through which nutrients and waste products can diffuse between a cell and the nearest blood vessel. In other words, almost all cells in the human body are generally less than that distance from some blood vessel. Accordingly the filler piece may be designed so that every point in the filler piece is within such a distance, such as 2 mm, of a channel or surface recess or similar feature. It is assumed that the channels and surface recesses in the filler piece may become the sites of blood vessels, as well as serving as pathways for early progression of tissue growth into the filler piece.

The channels may go through the filler piece from the one surface to another surface. In this situation, there is freedom to remove a substantial amount of material from the surface without obliterating the features, i.e., the channels would still be apparent even after such removal of material.

Alternatively, it is possible that the features could be dead-end recesses. If the features are dead-end recesses, the depth of the recesses determines how much material could be removed from the surface of the filler piece and still result in there being surface features such as recesses on the bone-facing surfaces. Accordingly, the depth of the surface features may be chosen to be deeper than any likely depth removal of material from bone-facing surfaces, so that there will still be features on the surface which is newly created by such removal of material.

The filler piece may have a carrying feature suitable to allow the filler piece to be gripped or carried by a carrying tool.

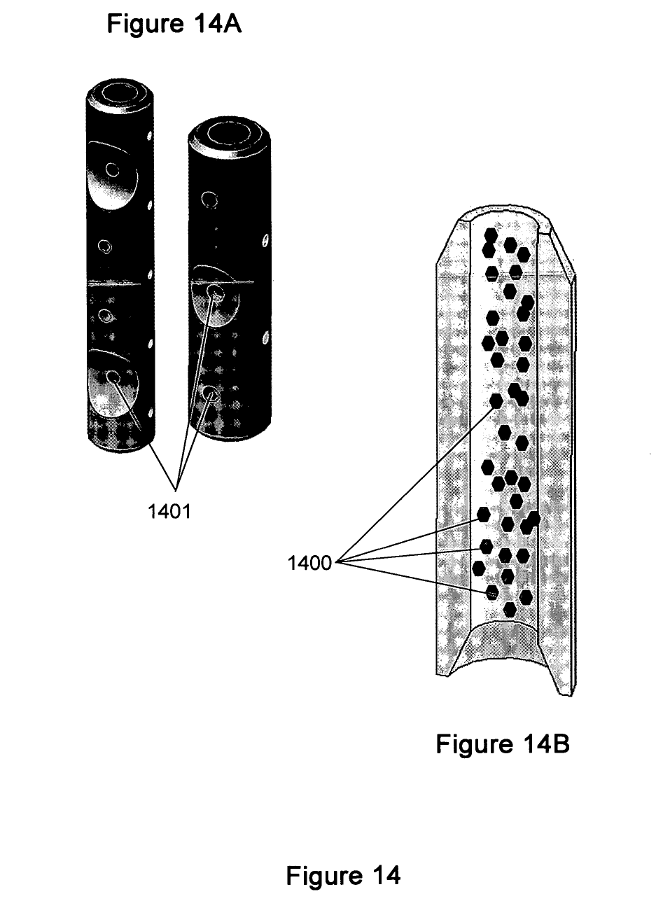

FIG. 14A shows a filler article (e.g., an insert) having holes 1401 according to an embodiment of the invention. For instance, the article may also comprise holes through the wall of the article in places as desired. These holes 1401 may serve as paths for the introduction of additional material to the interior of the article, or any other purpose. The holes 1401 may extend partially or completely through the article, which may have one or more inner cavities. The articles may comprise surface features such as indentations, patterning, etc, and may comprise internal passageways, channels, etc.

FIG. 14B shows a cross-sectional view of another article that shows DBM 1400 in the inner portion of the article.

The article may comprise a porous osteoconductive material such as any member of the calcium phosphate family, such as in particular beta tricalcium phosphate. The presence of such composition may improve the osteoconductivity of the article. The article may in general contain biocompatible materials of any composition. The material may be either resorbable or nonresorbable. The material may be porous. The porosity fraction, pore size distribution, etc. may be as described in co-pending commonly assigned U.S. Provisional Patent Application No. 60/466,884, filed Apr. 30, 2003, entitled “Bone void filler & method of manufacture,” and in U.S. application Ser. No. 10/837,541, filed Apr. 30, 2004, the disclosures of which are incorporated herein by reference in their entirety.

The article (e.g., spinal insert or filler piece) may further comprise (e.g., contain) particles of DBM. The DBM may or may not have a carrier fluid or gel or substance, e.g., in the interior of the article.

According to some embodiments of the invention, the invention may comprise a kit comprising a spinal cage insert constructed in accordance with the teachings herein together with one or more additional items useful with the spinal cage insert. The additional one or more items in the kit may include any of the following: a spinal cage appropriate for use with the spinal cage insert; tools for installation of the spinal cage insert into the spinal cage; tools for installation of the spinal cage into the patient; a spreader for spreading apart subcomponents of the spinal cage insert, if the spinal cage insert is so designed; putty, paste or adhesive suitable to adhere the spinal cage insert to the spinal cage; a filler piece, or more than one filler piece possibly of differing dimensions; any other appropriate tooling and/or templates suitable for modifying bone; and any other instruments or materials useful during surgery.

The described articles may be used in connection with spinal fusion (arthrodesis). In some spinal fusions, there is inter-vertebral fusion such as using spinal cages or other fixation or attachment hardware. The spinal cage insert as described elsewhere herein may be used to fill the interior of a spinal cage. It is possible that the spinal cage insert may be retained within the spinal cage by a putty, bone cement, paste, or other adhesive. Such materials are commonly used in the field of orthopedics to fill voids in bone fractures and surgery. Such a material may be placed between the spinal cage insert and the spinal cage, attaching the spinal cage insert and spinal cage together so as to create a single unit. Such a material may be either natural or synthetic in origin.

In some instances it may be desirable to build up bone adjacent to the vertebrae to achieve greater strength of the (finally healed) bone. The spinous process extends towards the posterior from the vertebrae. It is possible to add filler pieces alongside the spinous process on either side, e.g., between the spinous process and the transverse process. Alternatively, or in addition, it is possible to add a filler piece in the general vicinity of the spinous process, which may involve removing some of the spinous process. In any of these cases, the installed filler piece may replace material (if any) of the spinous process which may have been removed, and may also bridge between vertebrae. In any of these situations, the filler piece might not be expected to carry load between the vertebrae. Load-carrying could be achieved by spinal cages installed between the vertebrae and could also be achieved by instrumentation connecting the vertebrae in other places.

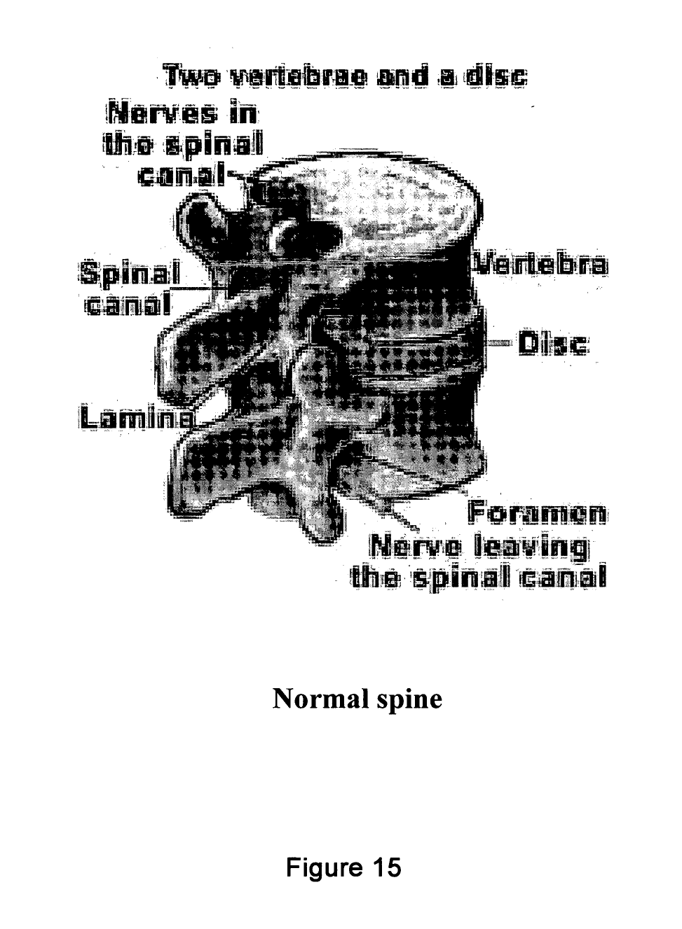

FIG. 15 shows a spinous process.

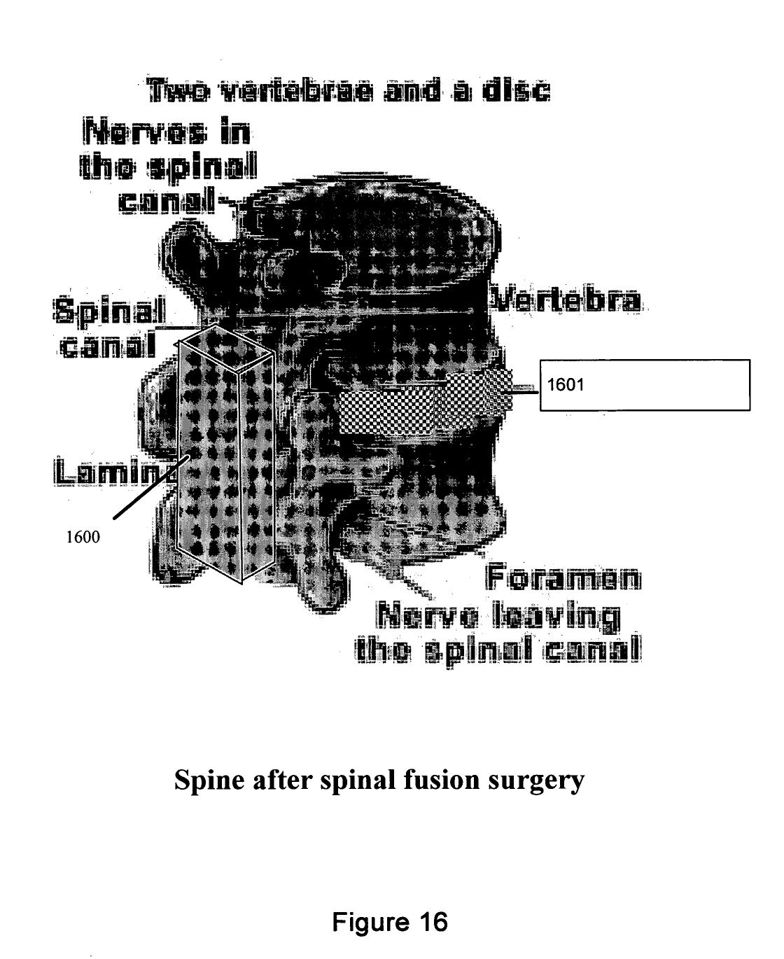

FIG. 16 illustrates use of a rectangular prismatic filler piece. The piece may be installed directly in the region of the spinous process. For instance, FIG. 16 may represent a portion of a spine after surgery. A strut 1600 comprising a filler article may be attached to the spine. The strut may be used to replace the spinous processes. As shown in the diagram, there may be inter-body fusion 1601 between two of the vertebrae.

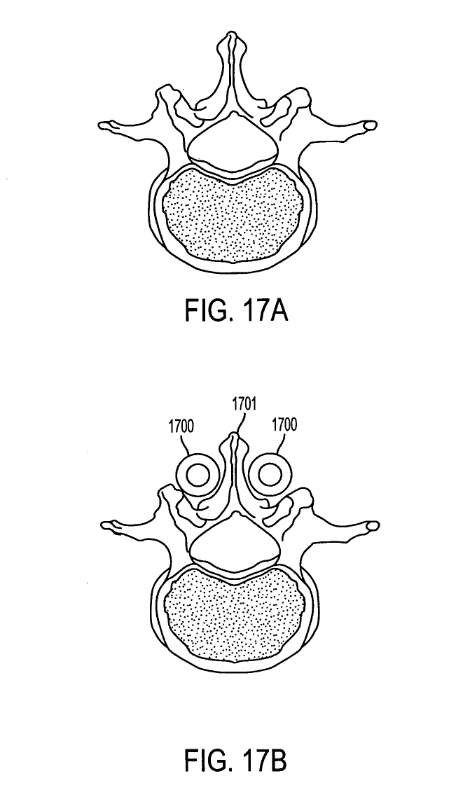

FIG. 17 illustrates use of filler pieces, in this case tubular filler pieces 1700. The filler pieces 1700 may be installed in the regions between the spinous process 1701 and the transverse process. The method of installation may also include, after the filler piece has been installed, shaping any exposed portion of the filler piece by removing material from it. For example, the exposed edge of the filler piece may be shaped by removal of material so as to match contours of native bone nearby. Such reshaping may be helpful in reducing the formation of adhesions between the filler piece and adjacent soft tissue, which can be painful for the patient.

Use of elongated tubular filler pieces of the present invention is illustrated in FIG. 17. In such a procedure, the articles 1700 of the present invention may be implanted adjacent to the spinous process 1701, on both sides of the spinous process 1701 (or on just one side if so desired). The spinous process 1701 itself may be either modified or unmodified. This is illustrated in FIG. 17A (prior to surgery) and 17B (after surgery adding the tubular filler pieces 1700).

Still another possibility is that the article of the present invention could be installed (alternatively or in addition) in regions adjacent to the transverse processes of vertebrae.

The surgical procedure may include installing a number of tubular filler pieces end-to-end to achieve the desired overall length of filler piece, and can include engaging the articles end-to-end. The surgical procedure can include fixturing (or otherwise affixing) the articles to hold them in the desired location.

The surgical procedure can also include placing an additional substance into the elongated tubular filler piece. For example, DBM or a composition containing DBM and other substances can be placed or injected into the interior of the article either before or after the article is in place in the patient's body.

Depending on the local shape of native bone, the as-manufactured surface of the filler piece may touch natural bone or other bone filler material. Alternatively, it is possible that the filler piece might require some removal of material from it before installation, such as to improve fit. For instance, a surgeon might use a scalpel or other tool to sculpt the article for a better fit, as needed. Some on-site minor tailoring of the article may be useful because each person's bones may be shaped slightly differently. Articles that do not couple directly to bone may not require any modification whatsoever, depending on the circumstances.

In any of this use of filler pieces, the use of spinal cages or other instrumentation may create a situation in which the filler pieces of the present invention, as installed, carry little or no mechanical load. This is appropriate due to the fact that the mechanical strength of the filler piece(s) themselves at the time of installation may be fairly limited. For instance, the filler pieces may break under 10 pounds weight or less. The method of installation may include soaking the spinal cage insert and/or the filler piece in blood, platelet rich plasma, bone marrow or other bodily fluids prior to final installation of the filler piece. Such soaking may help to promote ingrowth of natural bone.

The invention also includes aspects of methods of manufacture of spinal cage inserts (and filler pieces) according to embodiments of the invention. The manufacture of spinal cage inserts, struts, and other pieces will be described herein with reference to spinal cage inserts for the sake of simplicity. It should be appreciated that the systems and methods as described for spinal cage inserts may also be used for inserts and other filler pieces.

The method of manufacture may include three dimensional printing (“3DP”). 3DP provides the ability to precisely determine local geometric features and composition of a manufactured piece, to an extent that is not possible with most other manufacturing methods. Because the architecture or structure of the spinal cage insert (or other pieces) of the invention can be controlled through the use of three dimensional printing techniques, namely controlled particle packing with defined interparticle pores, good bone ingrowth is achievable once with optimal appropriate printing parameters. Furthermore, controlled, repeatable resportion characteristics and osteoconductivity are achieved. The spinal cage insert pieces of the invention eliminates substantial variability in tissue response due to the random distributions in pore size and internal structure.

Other forms of manufacturing, including but not limited to molding, could also be used in the manufacture of the described filler piece. The manufacturing method also may include a chemical reaction to form a desired substance, such as tricalcium phosphate, from precursors. The manufacturing method also may include the use of a decomposable porogen. Any of these aspects of the method may be used either separately or together in any combination.

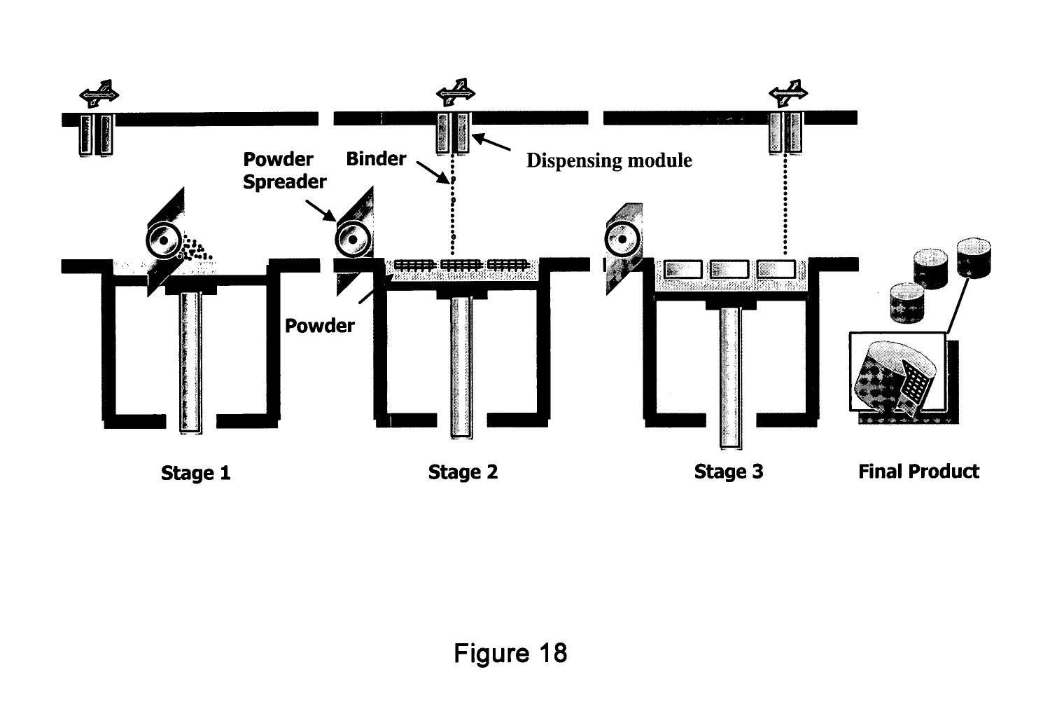

FIG. 18 shows a general schematic of a 3DP manufacturing process according to an embodiment of the invention. Three dimensional printing may include a set of steps which may be repeated as many times as are necessary to manufacture an piece. Three dimensional printing is described in U.S. Pat. Nos. 5,204,055 and 6,139,574, the disclosures of each of which are herein incorporated by reference in their entireties. At the beginning of the set of steps, powder may be deposited in the form of a layer. The powder may be deposited by roller-spreading or by other means such as slurry deposition.

In one aspect of the present invention, the deposited powder may comprise particles of precursors of a ceramic. Precursors may comprise hydroxyapatite and dicalcium phosphate, and even calcium pyrophosphate or other calcium-phosphorus compounds, as described elsewhere herein or in the incorporated references. The ceramic or precursor may in general include any member or members of the calcium phosphate family.

In another aspect of the invention, the deposited powder may comprise the desired ceramic. For example, the deposited ceramic may be tricalcium phosphate, and, in particular, may be β-tricalcium phosphate. The ceramic may be any other desired ceramic or mixture of ceramics.

In another method of manufacture of the invention, the deposited powder may comprise particles of a porogen, which may be decomposable. The proportion of the porogen to the other particles in the deposited powder may be chosen so as to result in a finished product having a desired porosity. The sizes and size distribution of the other particles (which may include ceramic and/or precursors) and the particles of the porogen may be chosen so as to determine the size and size distribution of the pores in the finished product. The porogen may be lactose, such as spray dried lactose, or another sugar, or in general, any substance which is capable of decomposing, into gaseous decomposition products, at a temperature which is permissible for the materials already in the product at the time of decomposition. This may be done with the other particles comprising either the desired ceramic, or precursors, or both. The average size of the particles of the porogen may be larger than the average size of the particles of the rest of the powder, and may even be significantly larger such as by a factor of approximately 5. For example, the size of the lactose particles may be on average about 120 to about 150 micrometers while the size of the other particles may be on average about 10 micrometers. The proportion of decomposable porogen to other substances may be, for example, about 0 to about 50% by weight. It has been found that a powder containing a combination of lactose and ceramic or precursors is easier to roller-spread than a powder containing only ceramic or precursors without lactose.

In still other aspects of the invention, the deposited powder may be or may include polymer particles or particles of demineralized bone matrix. Particles of demineralized bone matrix may be in an average size range of about 200 micrometers.

After the deposition of a powder layer, drops of a liquid may be deposited onto the powder layer to bind powder particles to each other and to other bound powder particles.

FIG. 18 shows a general schematic of a 3DP manufacturing process, where a powder layer is spread by a powder spreader, followed by dispensing of a binder liquid from a dispensing module. At each powder layer, timing of drop deposition such as from a printhead may be coordinated, for example by software, with the motion of the printhead in two axes, to produce a desired pattern of deposited droplets.

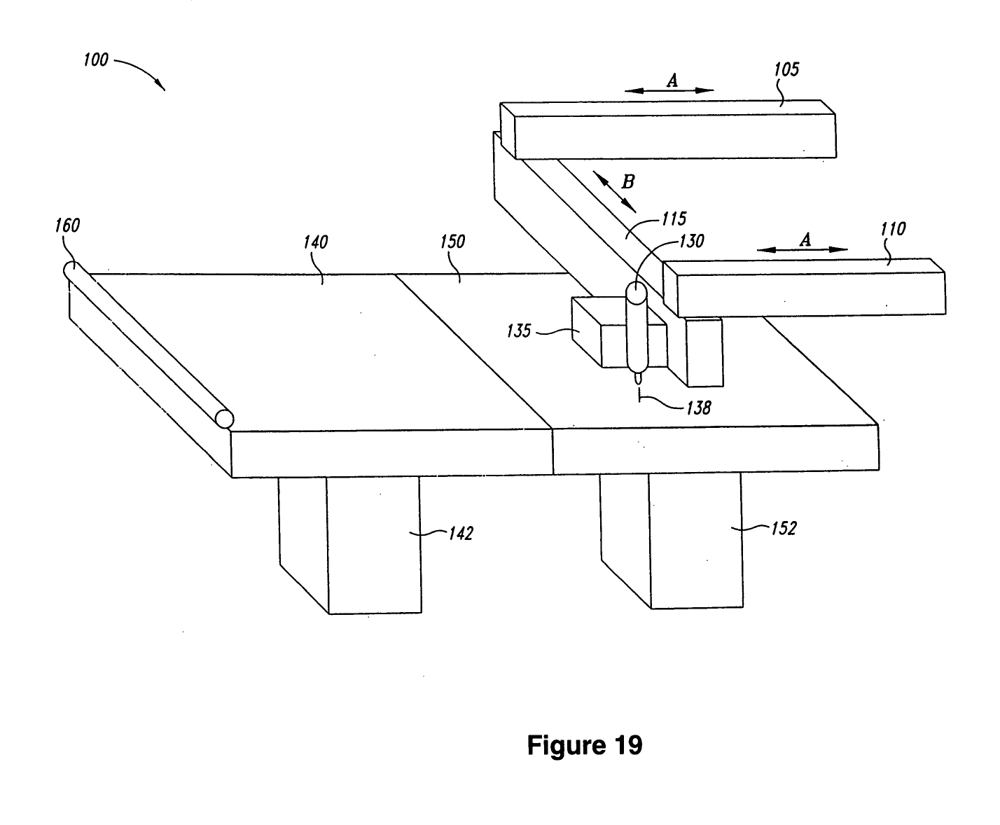

FIG. 19 provides a typical three-dimensional printing apparatus [100] in accordance with the prior art. The apparatus [100] includes a roller [160] for rolling powder from a feed bed [140] onto a build bed [150]. Vertical positioners [142 and 152, respectively] position the feed bed [140] and the build bed [150] respectively. Slow axis rails [105, 110] provide support for a printhead [130] in the direction of slow axis motion A, and fast axis rail [115] provides support for the printhead [130] in the direction of fast axis motion B. The printhead [130] is mounted on support [135], and dispenses liquid binder [138] onto the build bed [150] to form the three-dimensional object.

The term droplets will be understood to include not only spherical drops but any of the various possible dispensed fluid shapes or structures as are known in the art. The liquid may be dispensed by a dispensing device suitable for dispensing small quantities of liquid drops, which may resemble an ink-jet printhead. For example, the dispensing device could be a microvalve (The Lee Company, Essex, Conn.) or it could be a piezoelectric drop-on-demand printhead, a continuous-jet printhead, or any other type of printhead as is known in the art. The liquid may comprise a binding substance dissolved in a solvent, which may be water.

The binding substance may be capable of decomposing into gaseous decomposition products at a temperature that is permissible for the materials already in the product at the time of decomposition. The binding substance may, for example, be polyacrylic acid. In certain materials systems (such as demineralized bone matrix), the binder substance may be left in the finished product. In certain materials systems, such as polymers, the binder liquid may be a pure solvent.

After this liquid dispensing process is completed on one layer, another layer of powder may be spread and the liquid dispensing may be repeated, and so on until a complete three-dimensional object has been built. The printing pattern(s) in each printed layer may in general be different from the printing pattern(s) in other layers, with each printing pattern being chosen appropriately so as to form an appropriate portion of a desired piece. During 3DP printing, the unbound powder supports the bound shape and the later deposited layers of powder. At the end of the 3DP printing process the powder particles that are unbound and untrapped may be removed, leaving only the shape which has been bound together.

After separation of the bound shape from unbound powder, the bound shape may be processed with a heat treatment suitable to accomplish any one or more, or all, of several purposes. (For certain powder materials such as polymer and demineralized bone matrix, heat treatment may be impermissible.) The heating may be performed so as to thermally decompose the decomposable porogen (if used) so that the porogen exits the bound shape in the form of gaseous decomposition products. A typical decomposable porogen may decompose at temperatures below 400° C. The heating may also be performed so as to thermally decompose the binder substance so that the binder substance also exits the bound shape in the form of gaseous decomposition products. A typical temperature for this purpose may be about 400° C. If ceramic particles are used, the heating may also be performed so as to partially sinter the ceramic particles together, thereby forming a porous structure of ceramic particles bound directly to other ceramic particles. A typical temperature and duration for this purpose, for members of the calcium phosphate family, may be about 1100° C. to about 1300° C. for about one to about several hours, depending on the ceramic. The heating may also be performed so as to cause the reaction of precursors to form the desired final ceramic, if such materials are used. The described heating may be performed in an oven whose atmosphere is ordinary atmospheric air, or can be performed in another special atmosphere if needed.

The formation of a desired final ceramic from precursors can involve a chemical reaction. For example, hydroxyapatite, which is Ca₁₀(PO₄)₆(OH)₂, plus dicalcium phosphate, which is CaHPO₄, yields tricalcium phosphate, which is Ca₃(PO₄)₂. The following is an exemplary reaction as described above: Ca₅(PO₄)₃OH+CaHPO₄→2Ca₃(PO₄)₂+H₂O (i.e., Hydroxyapatite+Dibasic Calcium Phosphate=Tricalcium Phosphate+Water).

In one embodiment of the invention, the desired ceramic is produced from a specific combination of the following proportions, which involves adding calcium pyrophosphate in an amount of about 3% to the powder. The proportions are as follows: about 58.2% precursors, about 38.8% lactose, and about 3% calcium pyrophosphate. Calcium pyrophosphate is Ca₂P₂O₇. Further details of chemical reaction among calcium-phosphorus compounds are given in commonly assigned U.S. patent application Ser. No. 10/122,129, filed Apr. 12, 2002, the disclosure of which is herein incorporated by reference in its entirety. This reaction may take place at elevated temperatures such as about 1100 C or higher, depending on individual chemistry and time duration. The methods of the present invention may further include the formation of a reaction product, such as a ceramic such as tricalcium phosphate, from precursors, regardless of whether three dimensional printing is or is not used. The methods of the present invention can include the use of a decomposable porogen, regardless of whether three dimensional printing is used. The methods of the present invention can include the use of a decomposable binder substance, regardless of whether three dimensional printing is used.

For certain applications such as simple geometries, the spinal cage insert piece of the invention could also be manufactured by molding, or other appropriate methods. After any method of manufacturing the spinal cage insert piece of the invention, it is possible to apply one or more bioactive substances to the spinal cage insert piece of the invention such as by dispensing or dipping. The invention also includes a spinal cage insert piece of the invention manufactured by any of the described methods.

The invention also includes tooling and/or templates suitable for insuring that the final dimensions of the void closely match the as-manufactured dimensions of the spinal cage insert piece of the invention, for example to within a tolerance of about 0.5 millimeters. Either the cutting tool or the template may be coordinated with the pre-manufactured dimensions of the filler piece, either to insure a substantially exact fit or to insure a fit with a known amount of interference.

The tooling may include, for example, a rasp suitable to remove bone by abrading it. The tooling may include sharp-edged cutting tools, either hand-held or tools suitable to be driven by a powered driver, such as a rotary driver. The tooling may be tooling which itself determines the contour of the void, such as if the cutting is done all at once, or tooling which cuts smaller portions over numerous times and determines the contour of the voids as a result of the positions through which the tooling is moved.

The invention also includes a kit containing the filler piece, or more than one filler piece possibly of differing dimensions. The kit may further include one or more appropriate items such as tooling and/or templates suitable for assuring a close fit between the void and the filler piece with minimal modification to the filler piece. The additional item or items in the kit may include any one or more of the following: tools for installation of the spinal cage insert into the bone void; tools for creation of the bone void; a spreader for spreading apart subcomponents of the spinal cage insert, if the spinal cage insert is so designed; putty, paste or adhesive suitable to adhere the spinal cage insert to the adjacent bone or other tissue; and any other instruments or materials useful during surgery. The tools for creation of the bone void may be geometrically matched to the spinal cage insert so as to result in a desired fit or a desired gap or even a desired interference between the spinal cage insert and the bone void created using the tools.

The kit may further include bone putty or other substances that may be useful during surgery.

The invention also includes installing the spinal cage insert pieces of the invention in a bone void. Installation can include using a bone putty, adhesive, or other such substance to retain the spinal cage insert piece in place, and to help fill gaps. Such materials are commonly used in the field of orthopedics to fill voids in bone fractures and surgery. Such a material may be placed between the spinal cage insert piece of the invention and the bone void. Such a material may be either natural or synthetic in origin.

Installation can include forcing or tapping the spinal cage insert piece into place to create frictional fit within the bone void. Installation may also include forcing or tapping the spinal cage insert into place so as to crush or shear off certain features of the spinal cage insert, thereby creating a frictional restraint. This can be done with the spinal cage insert pieces of the invention which are either untapered or tapered. The use of insertion force resulting in possible localized crushing may be useful in helping to limit the flow of blood which often takes place from freshly cut bone.

The pieces of the present invention can be used for any of a variety of medical indications. The pieces of the present invention can be used to fill cylindrical defects, such as those left by a drill bit. They can be used to fill voids such as cylindrical or other axisymmetric voids made in the iliac crest to harvest bone graft. They can be used to fill cylindrical or other axisymmetric voids made in the femur and tibia (or other bones) during ligament reconstruction. They can be used to fill defects following removal of a cylindrical implant (e.g. a sliding hip screw). They can be used to fill voids when returning to graft a cylindrical or other axisymmetric defect after treatment of an infection. The described piece can also be used by a surgeon after performing a core decompression drilling of the femoral neck or any other bone for osteonecrosis.

The spinal cage insert piece may be used for treatment of a variety of medical indications including situations that may result from the donation of bone, from trauma, from any surgical removal of bone, or for any other reason.

In general, surface recesses or channels can be on any surface of the filler piece. The spinal cage insert pieces of the invention provide the benefits of ceramic as a material or provide the benefit of demineralized bone matrix as a material, while also providing a desired pre-manufactured shape. The invention also provides features such as surface recesses or channels, which are believed to promote the ingrowth of natural bone.

The above description of illustrated embodiments of the invention is not intended to be exhaustive or to limit the invention to the precise form disclosed. While specific embodiments of, and examples for, the invention are described herein for illustrative purposes, various equivalent modifications are possible within the scope of the invention, as those skilled in the relevant art will recognize. Aspects of the invention can be modified, if necessary, to employ the process, apparatuses and concepts of the various patents and applications described above to provide yet further embodiments of the invention. The various embodiments described above can be combined to provide further embodiments. These and other changes can be made to the invention in light of the above detailed description. In general, in the following claims, the terms used should not be construed to limit the invention to the specific embodiments disclosed in the specification and the claims, but should be construed to include all bone substitutes that operate under the claims. Accordingly, the invention is not limited by the disclosure, but instead the scope of the invention is to be determined entirely by the following claims.

Certain shaped spinal cage inserts and compositions, as well as methods of manufacturing the same were disclosed in U.S. Provisional Application No. 60/512,498, filed Oct. 17, 2003; U.S. Provisional Application No. 60/512,414, filed Oct. 17, 2003; U.S. Provisional Application No. 60/577,736, filed Jun. 7, 2004; and U.S. patent application Ser. No. 10/122,129, filed Apr. 12, 2002, the disclosures of each of which are herein incorporated by reference in their entireties.

Certain methods, systems and apparatuses for use in three-dimensional printing and for engineered regenerative biostructures were disclosed in U.S. patent application Ser. No. 10/122,129, filed Apr. 12, 2002; U.S. patent application Ser. No. 10/189,795, filed Jul. 3, 2002; U.S. patent application Ser. No. 10/190,333, filed Jul. 3, 2002; U.S. patent application Ser. No. 10/189,799, filed Jul. 3, 2002; U.S. patent application Ser. No. 10/189,166, filed Jul. 3, 2002; U.S. patent application Ser. No. 10/189,153, filed Jul. 3, 2002; and U.S. patent application Ser. No. 10/189,797, filed Jul. 3, 2002, the disclosures of each of which are herein incorporated by reference in their entireties.

Patent applications incorporated by reference include commonly assigned “Methods and apparatus for engineered regenerative biostructures such as hydroxyapatite substrates for bone healing applications,” U.S. application Ser. No. 10/122,129, docket number 900122.432; “Apparatus, systems and methods for use in three-dimensional printing,” docket number 900122.452-457, U.S. application Ser. Nos. 10/189,795; 10/190,333; 10/189,799; 10/189,166; 10/189,153; 10/189,797; and “Spinal cage insert and method of manufacture,” U.S. Application No. 60/466,884, docket number 900122.468P1. All patents, patent applications and publications referred to herein are incorporated by reference in their entirety.

The above description of illustrated embodiments of the invention is not intended to be exhaustive or to limit the invention to the precise form disclosed. While specific embodiments of, and examples for, the invention are described herein for illustrative purposes, various equivalent modifications are possible within the scope of the invention, as those skilled in the relevant art will recognize. Aspects of the invention can be modified, if necessary, to employ the process, apparatuses and concepts of the various patents and applications described above to provide yet further embodiments of the invention. The various embodiments described above can be combined to provide further embodiments. These and other changes can be made to the invention in light of the above detailed description. In general, in the following claims, the terms used should not be construed to limit the invention to the specific embodiments disclosed in the specification and the claims, but should be construed to include all bone substitutes that operate under the claims. Accordingly, the invention is not limited by the disclosure, but instead the scope of the invention is to be determined entirely by the following claims.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A spinal cage insert for a spinal cage, wherein the spinal cage insert has a shape of dimensions substantially equal to those of an interior of the spinal cage, and wherein the spinal cage insert comprises a member of the calcium phosphate family.

2. The spinal cage insert of claim 1, wherein the calcium phosphate comprises tricalcium phosphate.

3. The spinal cage insert of claim 1, wherein the spinal cage insert has pores which are suitable to wick bodily fluids.

4. The spinal cage insert of claim 1, wherein the spinal cage insert has pores which make up between 20% and 45% by volume of the entire spinal cage insert.

5. The spinal cage insert of claim 1, wherein the spinal cage insert has pores which have a pore size distribution which has a peak between 8 micrometers and 20 micrometers.

6. The spinal cage insert of claim 1, wherein the spinal cage insert has pores which have a mean pore size of approximately 60 micrometers.

7. The spinal cage insert of claim 1, wherein the spinal cage insert has pores which range from approximately 1 micrometer to approximately 300 micrometers.

8. The spinal cage insert of claim 1, wherein the spinal cage insert has at least one of recessed surface features and channels therethrough.

9. The spinal cage insert of claim 8, wherein the spinal cage has perforations or openings, and at least some of the at least one of recessed surface features and channels coincide with at least some of the perforations or openings in the spinal cage when the spinal cage insert is installed in the spinal cage.

10. The spinal cage insert of claim 9, wherein the spinal cage insert and the interior of the spinal cage are rotationally non-symmetric.

11. The spinal cage insert of claim 9, wherein the spinal cage insert and the interior of the spinal cage are rotationally symmetric.

12. The spinal cage insert of claim 1, wherein the spinal cage insert is suitable to be inserted into the spinal cage with a translational motion.

13. The spinal cage insert of claim 1, wherein the spinal cage insert is suitable to be inserted into the spinal cage with simultaneous translational and rotational motion, wherein the translational motion is along a length axis of the spinal cage insert, and the rotational motion is around the length axis of the spinal cage insert.

14. The spinal cage insert of claim 1, wherein the spinal cage insert is suitable to be inserted into the spinal cage with translational motion followed by rotational motion.

15. The spinal cage insert of claim 1, wherein the spinal cage insert fits into the spinal cage with a frictional fit.

16. The spinal cage insert of claim 1, wherein the spinal cage insert has a taper on at least one external surface suitable to frictionally engage the spinal cage.

17. The spinal cage insert of claim 1, wherein the spinal cage insert has at least one of ribs and protrusions.

18. The spinal cage insert of claim 17, wherein the at least one of ribs and protrusions interfere with the spinal cage when the spinal cage insert is in the spinal cage, and wherein the at least one of ribs and protrusions are crushable.

19. The spinal cage insert of claim 1, wherein the spinal cage insert comprises at least two parts which together are suitable to be inserted into and fit closely in the interior of the spinal cage.

20. The spinal cage insert of claim 1, wherein the spinal cage insert is suitable to be broken into at least two parts which together are suitable to be inserted into and fit closely in the interior of the spinal cage.

21. The spinal cage insert of claim 1, wherein the spinal cage insert has a chamfer or taper on at least one external surface usable to guide the spinal cage insert into the spinal cage.

22. The spinal cage insert of claim 1, wherein the spinal cage insert has at least one carrying feature suitable to allow the spinal cage insert to be at least one of gripped or carried by a tool.

23. The spinal cage insert of claim 1, wherein the spinal cage insert is suitable to be trapped inside the spinal cage by a locking component which couples to both the spinal cage and the spinal cage insert.

24. The spinal cage insert of claim 1, further comprising at least one bioactive substance.

25. The spinal cage insert of claim 1, wherein the spinal cage insert is made by methods which include three dimensional printing.

26. A spinal cage insert for a spinal cage, wherein the spinal cage insert has a shape suitable to be inserted into and fit closely in an interior of the spinal cage, and wherein the spinal cage insert comprises demineralized bone matrix.

27. The spinal cage insert of claim 26, wherein the spinal cage insert comprises pores.

28. The spinal cage insert of claim 27, wherein the pores are suitable to wick bodily fluids.

29. The spinal cage insert of claim 26, wherein the spinal cage insert has recessed surface features or channels therethrough.

30. The spinal cage insert of claim 26, further comprising at least one bioactive substance.

31. A spinal cage insert for a spinal cage, wherein the spinal cage insert has a shape suitable to be inserted into and fit closely in an interior of the spinal cage, and wherein the spinal cage insert has surface recesses or channels therethrough.

32. The spinal cage insert of claim 31, wherein the surface recesses or channels have a smallest dimension along a surface of the spinal cage insert, wherein the smallest dimension is in the range from approximately 50 micrometers to approximately 3000 micrometers.

33. The spinal cage insert of claim 32, wherein the spinal cage insert has pores.

34. The spinal cage insert of claim 33, wherein the pores are suitable to wick bodily fluids.

35. The spinal cage insert of claim 31, further comprising at least one bioactive substance.

36. The spinal cage insert of claim 26, wherein the spinal cage insert is made by methods which include three dimensional printing.

37. The spinal cage insert of claim 31, wherein the spinal cage insert is made by methods which include three dimensional printing.

38. The spinal cage insert of claim 31, wherein the spinal cage insert is made by methods which include molding.

39. The spinal cage insert of claim 26, wherein the spinal cage insert is made by methods which include molding.

40. The spinal cage insert of claim 1, wherein the spinal cage insert is made by methods which include molding.

41. A kit comprising the spinal cage insert of claim 1 and at least one item selected from the group consisting of:
a matched spinal cage;
tooling suitable for installing the spinal cage insert into the spinal cage;
tooling suitable for installing the spinal cage into a patient;
a spreader for spreading apart subcomponents of the spinal cage insert;
one or more other surgical instruments;
a substance suitable for adhering the spinal cage insert to the spinal cage; and
at least one other substance suitable for use during surgery.

42. A kit comprising the spinal cage insert of claim 25 and at least one item selected from the group consisting of:
a matched spinal cage;
tooling suitable for installing the spinal cage insert into the spinal cage;
tooling suitable for installing the spinal cage into a patient;
a spreader for spreading apart subcomponents of the spinal cage insert;
at least one other surgical instruments;
a substance suitable for adhering the spinal cage insert to the spinal cage; and
other substances suitable for use during surgery.

43. A kit comprising the spinal cage insert of claim 30 and at least one item selected from the group consisting of:
a matched spinal cage;
tooling suitable for installing the spinal cage insert into the spinal cage;
tooling suitable for installing the spinal cage into a patient;
a spreader for spreading apart subcomponents of the spinal cage insert; other surgical instruments;
a substance suitable for adhering the spinal cage insert to the spinal cage; and
at least one second substance suitable for use during surgery.

44. A method of manufacturing a spinal cage insert, the method comprising:
depositing a layer of powder having powder particles, wherein the powder particles comprise at least one member of the calcium phosphate family;
depositing onto the layer of powder in appropriate places a binder liquid suitable to bind powder particles other powder particles;
repeating the above steps as needed to form a bound shape which is a spinal cage insert; and
separating the bound shape from unbound powder.

45. The method of claim 44, wherein depositing the binder liquid is done in a pattern suitable to create a bound shape having surface recesses or internal channels.

46. The method of claim 44, wherein depositing the powder particles comprises depositing powder particles which have a mean size in the range of 20 micrometers to 40 micrometers.

47. The method of claim 44, further comprising after separating the bound shape from the unbound powder, heating the bound shape suitably to partially sinter the bound shape.

48. A method of manufacturing a spinal cage insert, the method comprising:
depositing a layer of powder comprising powder particles, wherein the powder particles comprise precursors of at least one member of the calcium phosphate family;
depositing onto the layer of powder in appropriate places a binder liquid suitable to bind powder particles to other powder particles;
repeating the above steps as needed to form a bound shape which is a spinal cage insert;
separating the bound shape from unbound powder; and
heating the bound shape suitably both to cause reaction between the precursors in the powder and to partially sinter the bound shape.

49. The method of claim 48, wherein depositing the powder particles comprises depositing powder particles which have a mean size in the range of approximately 10 micrometers.

50. The method of claim 48, further comprising heating the bound shape to a temperature sufficient to cause the precursors to react to form a desired ceramic.

51. The method of claim 48, wherein the precursors react to form a desired ceramic at a temperature between 1100 C and 1300 C.

52. The method of claim 48, wherein the precursors comprise hydroxyapatite and dicalcium phosphate.

53. The method of claim 48, wherein depositing the powder particles comprises depositing powder particles which further comprise a decomposable porogen.

54. The method of claim 53, wherein the particles of the precursors have a first average particle size, and the particles of the decomposable porogen have a second average particle size, and the second average particle size is at least approximately 5 times as large as the first particle size.

55. A method of manufacturing a spinal cage insert, the method comprising:
depositing a layer of powder comprising powder particles, wherein the powder particles comprise particles of demineralized bone matrix;
depositing onto the layer of powder in appropriate places a binder liquid suitable to bind powder particles to other powder particles;
repeating the above steps as needed to form a bound shape which is a spinal cage insert; and
separating the bound shape from unbound powder.

56. The method of claim 55, wherein depositing the binder liquid is done in a pattern suitable to create a bound shape having surface recesses or internal channels.

57. The method of claim 55, wherein depositing the powder particles comprises depositing powder particles which have a mean size in the range of approximately 200 micrometers.

58. A method of making a spinal cage insert, the method comprising:
depositing a layer of a powder comprising particles of a biocompatible substance and particles of a decomposable porogen suitable to decompose into gaseous decomposition products;
depositing onto the layer of powder in appropriate places a binder liquid suitable to bind powder particles other powder particles;
repeating the above steps as needed to form a bound shape which is a spinal cage insert;
separating the bound shape from unbound powder; and
heating the bound shape to a temperature sufficient to thermally decompose the decomposable porogen into gaseous decomposition products.

59. The method of claim 58, wherein the particles of the biocompatible substance have a first average particle size, and the particles of the decomposable porogen have a second average particle size, and the second average particle size is at least 5 times as large as the first particle size.

60. The method of claim 58, wherein the biocompatible substance is tricalcium phosphate.

61. The method of claim 58, wherein the porogen comprises lactose or another sugar.

62. A method of making a spinal cage insert, the method comprising:
forming powder into a desired shape, wherein the powder comprises precursors suitable to react to form a desired ceramic comprised in a spinal cage insert; and
heating the precursors to a temperature suitable to cause the precursors to react.

63. A method of making a spinal cage insert, the method comprising:
forming powder into a desired shape, wherein the powder comprises a decomposable porogen; and
heating the powder to a temperature suitable to decompose the decomposable porogen, wherein the decomposed porogen is comprised in a spinal cage insert.

64. A spinal cage insert for a spinal cage, wherein the spinal cage insert has a shape of dimensions substantially equal to those of an interior of the spinal cage, and wherein the spinal cage insert comprises a member of the calcium phosphate family, and wherein the spinal cage insert further comprises an internal cavity.

65. The spinal cage insert of claim 64, further comprising particles of demineralized bone contained inside the internal cavity.

66. The spinal cage insert of claim 65, further comprising a cap suitable to close the internal cavity.

67. The spinal cage insert of claim 66, wherein the cap comprises gelatin.

68. The spinal cage insert of claim 67, wherein the gelatin is in a dried state.

69. The spinal cage insert of claim 67, wherein the gelatin is in a gel state.

70. The spinal cage insert of claim 66, wherein the cap comprises gelatin which partially interpenetrates the spinal cage insert.

71. A bone filler piece which comprises a structure which is osteoconductive and which defines at least one macroscopic internal feature, wherein at least one macroscopic internal feature contains demineralized bone matrix.

72. The filler piece of claim 71, wherein the structure comprises beta tricalcium phosphate.

73. The filler piece of claim 71, wherein the structure comprises pores having an average pore dimension, and wherein at least one macroscopic internal feature has all dimensions greater than three times the average pore dimension.

74. The filler piece of claim 71, wherein at least one macroscopic internal feature has all dimensions greater than approximately 100 micrometers.

75. The filler piece of claim 71, wherein at least one macroscopic interior feature is accessible to an exterior of the filler piece.

76. The filler piece of claim 71, wherein at least one macroscopic internal feature is selected from the group consisting of a channel, a groove and an internal cavity.

77. The filler piece of claim 71, wherein at least one macroscopic internal feature, having feature internal dimensions, is connected to an exterior of the filler piece by a channel whose internal cross-section dimensions are smaller than the feature internal dimensions.

78. The filler piece of claim 71, wherein at least a majority of the demineralized bone matrix exists in the form of particles having all of their dimensions greater than approximately 100 micrometers.

79. The filler piece of claim 71, wherein the structure comprises pores having pore sizes between 1 micrometer and 1000 micrometers.

80. The filler piece of claim 71, wherein the structure comprises a single piece.

81. The filler piece of claim 71, wherein the structure comprises two or more pieces suitable to be joined together.

82. The filler piece of claim 81, wherein at least one of the pieces has a closed end.

83. The filler piece of claim 71, further comprising a cap suitable to close the macroscopic internal feature.

84. The filler piece of claim 83, wherein the cap comprises dried gelatin.

85. The filler piece of claim 83, wherein the cap comprises gelatin in a gel state.

86. The filler piece of claim 71, wherein the structure comprises particles partially joined to other particles.

87. The filler piece of claim 86, wherein the particles are joined to other particles by necks having a composition which is substantially the same as the composition of the particles.

88. The filler piece of claim 86, wherein the particles are joined to to other particles by necks having a composition which is different from the composition of the particles.

89. The filler piece of claim 71, wherein the filler piece further comprises, in at least some space not occupied by any other materials, a third material.

90. The filler piece of claim 71, wherein the filler piece is configured suitably to augment a vertebra or vertebrae of the spine.

91. A filler piece for augmenting exteriors of vertebrae, comprising a porous osteoconductive ceramic material shaped suitably to fit adjacent to more than one vertebrae.

92. The filler piece of claim 91, wherein the filler piece is shaped to fit between a spinous process and a transverse process of a vertebra.

93. The filler piece of claim 91, wherein the filler piece is shaped to fit in place of at least a portion of a spinous process of a vertebra.

94. The filler piece of claim 91, wherein the filler piece is shaped to fit between a spinous process and a transverse process of more than one vertebrae.

95. The filler piece of claim 91, wherein the filler piece is shaped to fit in place of at least a portion of a spinous process of more than one vertebrae.

96. The filler piece of claim 91, wherein the filler piece comprises more than one individual piece suitable to fit into each other.

97. The filler piece of claim 96, wherein one filler piece comprises a concave chamfer and a neighboring filler piece comprises a convex chamfer.

98. The filler piece of claim 96, wherein one filler piece comprises a post and a neighboring filler piece comprises a cylindrical empty space suitable to receive the post or a step, wherein one filler piece comprises a post or a step, and a neighboring filler piece comprises a cylindrical empty space suitable to receive the post or step.

99. The filler piece of claim 96, wherein a first filler piece comprises teeth and a neighboring filler piece comprises teeth configured to engage the teeth on the first filler piece.

100. The filler piece of claim 91, wherein the filler piece comprises at least one internal cavity suitable for containing an osteoinductive material.

101. The filler piece of claim 100, further comprising a cap suitable to close the internal cavity.

102. The filler piece of claim 100, wherein the cap comprises gelatin.

103. The filler piece of claim 100, wherein the gelatin interpenetrates with a portion of the filler piece.

104. The filler piece of claim 91, wherein the filler piece further comprises an osteoinductive material.

105. The filler piece of claim104, wherein the osteoinductive material comprises demineralized bone matrix.

106. A method of manufacturing a filler piece, the method comprising:
manufacturing a structure which is osteoconductive and which comprises macroscopic internal features; and
depositing into at least some of the macroscopic internal features a composition comprising particles of osteoinductive material;
closing the macroscopic internal features suitably to contain the particles of osteoinductive material.

107. The method of claim 106, wherein closing the macroscopic internal features comprises applying a cap.

108. The method of claim 106, wherein closing the macroscopic internal features comprises applying a cap which comprises gelatin.

109. The method of claim 106, wherein applying the cap comprises interpenetrating a fluid material into a portion of the structure.

110. The method of claim 106, further comprising, after applying the cap, drying the cap.

111. The method of claim 106, wherein manufacturing the structure comprises manufacturing a structure which has both macroscopic internal features of suitable size for depositing demineralized bone matrix and macroscopic internal features which are too small for depositing demineralized bone matrix.

112. The method of claim 106, wherein manufacturing the structure comprises manufacturing a sturcture in two or more parts suitable to assemble to form the filler piece.

113. The method of claim 106, wherein manufacturing the structure comprises three dimensional printing.

114. The method of claim 106, wherein manufacturing the structure comprises molding.

115. The method of claim 106, wherein manufacturing the structure comprises removing material.

116. The method of claim 106, wherein manufacturing the structure comprises three dimensional printing onto a powder which comprises a porogen which is suitable to decompose into gaseous decomposition products at a suitable temperature.

117. The method of claim 106, wherein manufacturing the structure comprises three dimensional printing onto a powder which comprises precursors suitable to react to form a desired ceramic substance.

118. The method of claim 106, wherein the powder comprises precursors suitable to react to form tricalcium phosphate and also comprises an amount of calcium pyrophosphate.

119. The method of claim 106, further comprising, after all the listed steps, infiltrating another substance into space not occupied by any other substance.

120. A filler piece made by the method of claim 106.

121. A method of augmenting exteriors of vertebrae, comprising:
mechanically anchoring adjacent vertebrae to each other; and
installing, adjacent to external surfaces of vertebrae, a filler piece comprising a synthetic porous osteoconductive material having a definite shape suitable to cause bone to grow joining the adjacent vertebrae to each other.

122. The method of claim 121, wherein installing the filler piece comprises installing the filler piece in space between a spinous process and a transverse process.

123. The method of claim 121, wherein installing the filler piece comprises removing bone from the vertebrae.

124. The method of claim 121, wherein installing the filler piece comprises installing the filler piece in a region normally occupied by a spinous process.

125. The method of claim 121, wherein installing the filler piece comprises installing a filler piece which further comprises osteoinductive material.

126. The method of claim 121, wherein the osteoinductive material comprises demineralized bone matrix.

127. The method of claim 121, wherein the filler piece comprises an internal cavity containing demineralized bone matrix.

128. The method of claim 127, wherein the filler piece further comprises a cap suitable to close the internal cavity.

129. The method of claim 128, wherein the cap comprises gelatin.

130. The methods of any one of claims 44, 62, 63, and 121, wherein the powder comprises precursors of at least one member of the calcium phosphate family, and wherein the powder comprises a composition in the proportions of about 58.2% precursors, about 38.8% lactose, and about 3% calcium pyrophosphate.
